SEQUENCE LISTING (28 sequences)

SEQ ID NO: 1 - protein, 17 aa, synthetic construct
KSSQSVLLSANQKNYLA

SEQ ID NO: 2 - protein, 7 aa, synthetic construct
WASTRES

SEQ ID NO: 3 - protein, 9 aa, synthetic construct
HQGYTLPWT

SEQ ID NO: 4 - protein, 17 aa, synthetic construct
KSSQSVLLSANQKNYLA

SEQ ID NO: 5 - protein, 7 aa, synthetic construct
WASTRES

SEQ ID NO: 6 - protein, 9 aa, synthetic construct
HQGYTLPWT

SEQ ID NO: 7 - protein, 5 aa, synthetic construct
FFGIH

SEQ ID NO: 8 - protein, 17 aa, synthetic construct
FTSRDGNDKYYADSVKG

SEQ ID NO: 9 - protein, 7 aa, synthetic construct
GNLAFDI

SEQ ID NO: 10 - protein, 7 aa, synthetic construct
GFSFSFF

SEQ ID NO: 11 - protein, 6 aa, synthetic construct
SRDGND

SEQ ID NO: 12 - protein, 7 aa, synthetic construct
GNLAFDI

SEQ ID NO: 13 - protein, 113 aa, synthetic construct
DIVMTQSPDSLTASAGERATINCKSSQSVLLSANQKNYLAWYQQKPGQSPRLLIYWASTRESGVPDRFSGSGSGTDFTLTISNLQPEDAAVYYCHQGYTLPWTFGSGTKVEIK

SEQ ID NO: 14 - protein, 116 aa, synthetic construct
QVQLVQSGGGVVQPGRSLRLSCAASGFSFSFFGIHWIRQAPGKGLEWVAFTSRDGNDKYYADSVKGRFTISKDNSKNTVYLQMNSLRAEDTAVYYCGRGNLAFDIWGEGTLVTVSS

SEQ ID NO: 15 - protein, 220 aa, synthetic construct
DIVMTQSPDSLTASAGERATINCKSSQSVLLSANQKNYLAWYQQKPGQSPRLLIYWASTRESGVPDRFSGSGSGTDFTLTISNLQPEDAAVYYCHQGYTLPWTFGSGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 444 aa, synthetic construct
QVQLVQSGGGVVQPGRSLRLSCAASGFSFSFFGIHWIRQAPGKGLEWVAFTSRDGNDKYYADSVKGRFTISKDNSKNTVYLQMNSLRAEDTAVYYCGRGNLAFDIWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 17 - protein, 444 aa, synthetic construct
QVQLVQSGGGVVQPGRSLRLSCAASGFSFSFFGIHWIRQAPGKGLEWVAFTSRDGNDKYYADSVKGRFTISKDNSKNTVYLQMNSLRAEDTAVYYCGRGNLAFDIWGEGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPDVFLFPPKPKDTLMISRTPEVTCVVVDVSHEDPEVKFNWYVDGVEVHNAKTKPREEQYNSTYRVVSVLTVLHQDWLNGKEYKCKVSNKALPAPEEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 18 - DNA, 339 bp, synthetic construct
  source 1..339: mol_type = other DNA
gatattgtgatgactcagagccccgactccctgactgcatctgcaggagagagggccaccatcaattgtaagtccagccagagtgtgttattgagtgccaaccagaaaaactacttagcttggtaccagcagaaacctggacagtctcctaggctgctcatctactgggcatccacccgagaatcgggggttcctgatcgattcagcggcagtgggtccggaacagatttcactcttaccatcagcaacctccagccggaagacgcggcagtgtattactgccaccagggatatactcttccatggacattcggcagtgggaccaaggtggagatcaaa

SEQ ID NO: 19 - DNA, 348 bp, synthetic construct
  source 1..348: mol_type = other DNA
caggtgcagctggtgcaatctgggggaggcgtggtccagcctggaaggtccctcagactctcctgtgcagcctctggattcagcttttccttcttcggcattcactggatccgccaggctccaggcaagggcctggagtgggtggcatttacatcaagagatggaaatgacaaatactatgcagactcagtgaagggccgattcacaatctccaaagacaactccaagaacactgtgtatctgcaaatgaacagcctgagagctgaggacacggcggtctattactgtggaaggggaaatttggcttttgacatctggggcgaagggaccctggtcaccgtctcgagt

SEQ ID NO: 20 - DNA, 660 bp, synthetic construct
  source 1..660: mol_type = other DNA
gatattgtgatgactcagagccccgactccctgactgcatctgcaggagagagggccaccatcaattgtaagtccagccagagtgtgttattgagtgccaaccagaaaaactacttagcttggtaccagcagaaacctggacagtctcctaggctgctcatctactgggcatccacccgagaatcgggggttcctgatcgattcagcggcagtgggtccggaacagatttcactcttaccatcagcaacctccagccggaagacgcggcagtgtattactgccaccagggatatactcttccatggacattcggcagtgggaccaaggtggagatcaaacgtacggtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 21 - DNA, 1332 bp, synthetic construct
  source 1..1332: mol_type = other DNA
caggtgcagctggtgcaatctgggggaggcgtggtccagcctggaaggtccctcagactctcctgtgcagcctctggattcagcttttccttcttcggcattcactggatccgccaggctccaggcaagggcctggagtgggtggcatttacatcaagagatggaaatgacaaatactatgcagactcagtgaagggccgattcacaatctccaaagacaactccaagaacactgtgtatctgcaaatgaacagcctgagagctgaggacacggcggtctattactgtggaaggggaaatttggcttttgacatctggggcgaagggaccctggtcaccgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaaaagcctctccctgtccccg

SEQ ID NO: 22 - DNA, 1332 bp, synthetic construct
  source 1..1332: mol_type = other DNA
caggtgcagctggtgcaatctgggggaggcgtggtccagcctggaaggtccctcagactctcctgtgcagcctctggattcagcttttccttcttcggcattcactggatccgccaggctccaggcaagggcctggagtgggtggcatttacatcaagagatggaaatgacaaatactatgcagactcagtgaagggccgattcacaatctccaaagacaactccaagaacactgtgtatctgcaaatgaacagcctgagagctgaggacacggcggtctattactgtggaaggggaaatttggcttttgacatctggggcgaagggaccctggtcaccgtctcgagtgctagcaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccggacgtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccgaggagaaaaccatctccaaagccaaagggcagccccgagaaccacaggtgtacaccctgcccccatcccgggacgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaaaagcctctccctgtccccg

SEQ ID NO: 23 - protein, 417 aa, synthetic construct
APRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPKNISATRNGSQAFVHWQEPRAPLQGTLLGYRLAYQGQDTPEVLMDIGLRQEVTLELQGDGSVSNLTVCVAAYTAAGDGPWSLPVPLEAWRPVKEPSTPAFSWPWW

SEQ ID NO: 24 - protein, 426 aa, synthetic construct
APRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVLPQQPRNLHLVSRQPTELEVAWTPGLSGIYPLTHCTLQAVLSDDGMGIQAGEPDPPEEPLTSQASVPPHQLRLGSLHPHTPYHIRVACTSSQGPSSWTHWLPVETPEGVPLGPPKNISATRNGSQAFVHWQEPRAPLQGTLLGYRLAYQGQDTPEVLMDIGLRQEVTLELQGDGSVSNLTVCVAAYTAAGDGPWSLPVPLEAWRPVKEPSTPAFSWPWWGGGHHHHHH

SEQ ID NO: 25 - protein, 363 aa, synthetic construct
APRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLGGGGSGGGGSGGGGSGGGGSAAAAATCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSPGK

SEQ ID NO: 26 - protein, 335 aa, synthetic construct
PYFLEEPEDRTVAANTPFNLSCQAQGPPEPVDLLWLQDAVPLATAPGHGPQRSLHVPGLNKTSSFSCEAHNAKGVTTSRTATITVGGGGSGGGGSGGGGSGGGGSAAAAATCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSPGK

SEQ ID NO: 27 - protein, 448 aa, synthetic construct
APRGTQAEESPFVGNPGNITGARGLTGTLRCQLQVQGEPPEVHWLRDGQILELADSTQTQVPLGEDEQDDWIVVSQLRITSLQLSDTGQYQCLVFLGHQTFVSQPGYVGLEGLPYFLEEPEDRTVAANTPFNLSCQAQGPPEPVDLLWLQDAVPLATAPGHGPQRSLHVPGLNKTSSFSCEAHNAKGVTTSRTATITVGGGGSGGGGSGGGGSGGGGSAAAAATCPQCPAPELPGGPSVFVFPPKPKDVLSISGRPEVTCVVVDVGKEDPEVNFNWYIDGVEVRTANTKPKEEQFNSTYRVVSVLPIQHQDWLTGKEFKCKVNNKALPAPIERTISKAKGQTREPQVYTLAPHREELAKDTVSVTCLVKGFYPADINVEWQRNGQPESEGTYANTPPQLDNDGTYFLYSKLSVGKNTWQRGETLTCVVMHEALHNHYTQKSISQSPGK

SEQ ID NO: 28 - protein, 472 aa, synthetic construct
EESPFVGNPGNITGARGLTGTLRCQLQVQGEPPEVHWLRDGQILELADSTQTQVPLGEDEQDDWIVVSQLRIASLQLSDAGQYQCLVFLGHQNFVSQPGYVGLEGLPYFLEEPEDRTVAANTPFNLSCQAQGPPEPVDLLWLQDAVPLATAPGHGPQRNLHVPGLNKTSSFSCEAHNAKGVTTSRTATITVLPQQPRNLHLVSRQPTELEVAWTPGLSGIYPLTHCTLQAMLSDNEVGIQAGEPDPPEEPLTLQASVPPHQLRLGSLHPHTPYHIRVACTSSQGPSSWTHWLPVETPEGVPLGPPENISATRNGSQAFVHWQEPRAPLQGTLLGYRLAYQGQDTPEVLMDIGLRQEVTLELQGDGSVSNLTVCVAAYTAAGDGPWSLPVPLEAWRPGQAQPVHQLESWIQGLIHDLFLVPPIVKETSAPAFSWPEGGGSLVPRGSAAAGGGESHHHHHHGDILDYKDDDDKV

(19) United States
(12) Patent Application Publication
(10) Pub. No.: US 2026/0265384 A1
(43) Pub. Date: Sep. 10, 2026

(54) MONOCLONAL ANTIBODY OR ANTIGEN-BINDING FRAGMENT THEREOF THAT SPECIFICALLY BINDS TO AXL, AND USE THEREOF

(71) Applicant: JOINT STOCK COMPANY "BIOCAD", Saint Petersburg (RU)

(72) Inventors: Aleksandr Andreevich GORDEEV, G. Moscow (RU); Mariia Yurievna PUCHKOVA, g. Tula (RU); Iuliia Vladimirovna PUTINTCEVA, G. Moscow (RU); Iana Voldemarovna PURVINSH, d. Petrovka (RU); Mariia Denisovna DMITRIEVA, G. Novosibirsk (RU); Alina Sergeevna KOCHETKOVA, p. Vorobyevsky (RU); Elena Vladimirovna VINOGRADOVA, Saint Petersburg (RU); Dmitry Valentinovich MOROZOV, Saint-Petersburg (RU)

(21) Appl. No.: 19/490,084

(22) PCT Filed: May 22, 2024
(86) PCT No.: PCT/RU2024/050107

§ 371 Date: Dec. 4, 2025

(30) Foreign Application Priority Data
Jun. 6, 2023 (RU) 2023114863

(51) Int. Cl. C07K 16/28; A61K 39/00; A61K 45/06; A61P 35/00; C12N 15/85

(52) U.S. Cl. CPC C07K 16/2863; A61K 45/06; A61P 35/00; C12N 15/85; A61K 2039/505; C07K 2317/565; C07K 2317/732; C07K 2317/76; C07K 2317/92; C07K 2317/94

(57) ABSTRACT

The present invention relates to the field of biotechnology and medicine, in particular to a monoclonal antibody or antigen-binding fragment thereof that specifically binds to AXL. The invention further relates to nucleic acids encoding said antibody, expression vectors, host cells and methods for producing same, methods for producing the antibodies according to the invention, pharmaceutical compositions comprising the antibody according to the invention, pharmaceutical compositions comprising the antibody according to the invention and other therapeutically active compounds, methods for treating AXL-mediated diseases or disorders, uses of the antibodies or pharmaceutical compositions thereof for treating AXL-mediated diseases or disorders, and uses of the antibodies and other therapeutically active compounds for treating AXL-mediated diseases or disorders.

### FIELD OF THE INVENTION

The present invention relates to the field of biotechnology and medicine, in particular to a monoclonal antibody or antigen-binding fragment thereof that specifically binds to AXL. The invention further relates to nucleic acids encoding said antibody, expression vectors, host cells and methods for producing same, methods for producing the antibodies according to the invention, pharmaceutical compositions comprising the antibody according to the invention, pharmaceutical compositions comprising the antibody according to the invention and other therapeutically active compounds, methods for treating AXL-mediated diseases or disorders, uses of the antibodies or pharmaceutical compositions thereof for treating AXL-mediated diseases or disorders, and uses of the antibodies and other therapeutically active compounds for treating AXL-mediated diseases or disorders.

### BACKGROUND OF THE INVENTION

Monoclonal antibodies in the form of chimeric, humanized or fully human molecules have proven to be useful as effective medicine for treating multiple disorders and diseases.

Tyrosine kinase receptor Axl (ARK, TYRO7 and UFO) belongs to the tumor-associated macrophage (TAM) receptor family comprised of TYRO-3, AXL and MER. Structurally, TAM receptors comprise two immunoglobulin-like (Ig) domains, two portions of fibronectin type III (FNIII) in the extracellular domain, and the conserved amino acid sequence KW(I/L)A(I/L)ES in the kinase domain thereof. Gas6 (growth arrest specific 6) is a ligand for Axl. Axl, activated by Gas6 binding, propagates the signal through phosphorylation.

AXL has been shown to be overexpressed in various malignancies. Overexpression or activation of AXL is closely related to cell proliferation, survival, migration and invasion by means of activation of oncogenic signaling pathways, including the PI3K/Akt and/or MAPK/Erk pathways. Based on the above, AXL is a promising target in antitumor treatment.

AXL has been found to be overexpressed in many cancers, including lung, liver, kidney, colon, stomach, ovarian, pancreatic cancers, and glioblastoma.

Patent documents WO2015193428, WO2015193430, WO2017220695, WO2017180842 provide various antibodies to AXL.

There is a need to create novel antibodies that specifically bind to AXL and have high AXL antigen-binding affinity parameters.

### DISCLOSURE OF THE INVENTION

The authors of the present group of inventions have developed antibodies that specifically bind to AXL and have high AXL-binding affinity parameters. The authors of the present group of inventions have surprisingly developed antibodies that specifically bind to the second immunoglobulin-like domain of AXL (AXL-Ig2), to an AXL fragment comprising the first and second immunoglobulin-like domains of AXL (AXL-Ig1-Ig2), as well as to the complete extracellular portion of AXL (ExcAXL), but do not bind individually to the first immunoglobulin-like domain of AXL (AXL-Ig1). The antibodies to AXL according to the present invention inhibit AXL and GAS6 binding leading to the activation of AXL-mediated cellular signaling. The antibodies to AXL according to the present invention exhibit the properties of antibody-dependent cellular cytotoxicity (ADCC) and antibody-dependent cellular phagocytosis (ADCP). The antibodies to AXL according to the present invention have high aggregation stability and high stability in human blood serum. Furthermore, the antibodies to AXL according to the present invention exhibit antitumor activity and do not exhibit toxicity and local irritant effects.

### DEFINITIONS AND GENERAL METHODS

Unless defined otherwise herein, all technical and scientific terms used in connection with the present invention will have the same meaning as is commonly understood by those skilled in the art.

Furthermore, unless otherwise required by context, singular terms shall include plural terms, and the plural terms shall include the singular terms. Typically, the present classification and methods of cell culture, molecular biology, immunology, microbiology, genetics, analytical chemistry, organic synthesis chemistry, medical and pharmaceutical chemistry, as well as hybridization and chemistry of protein and nucleic acids described herein are well known by those skilled and widely used in the art. Enzyme reactions and purification methods are performed according to the manufacturer's guidelines, as is common in the art, or as described herein.

The term “KD” in this description refers to the affinity constant (or equilibrium constant) which is calculated from the ratio of Kd to Ka (i.e. Kd/Ka), and it is expressed as a molar concentration (M).

“Binding affinity” typically refers to the strength of the sum total of noncovalent interactions between a single binding site of a molecule (e.g. an antibody) and its binding partner (e.g. an antigen). Unless indicated otherwise, “binding affinity” refers to intrinsic (characteristic, true) binding affinity which reflects a 1:1 interaction between members of a binding pair (e.g. antibody and antigen). The affinity of a molecule X for its binding partner Y can generally be represented by the affinity constant (KD). The preferred Kd value is about 200 nM, 150 nM, 100 nM, 60 nM, 50 nM, 40 nM, 30 nM, 20 nM, 10 nM, 8 nM, 6 nM, 4 nM, 2 nM, 1 nM, or less. Affinity can be measured by common methods known in the art, including those described in the present description. Low-affinity antibodies typically bind an antigen slowly and tend to dissociate readily, whereas high-affinity antibodies typically bind an antigen faster and tend to remain bound longer. A variety of methods for measuring binding affinity are known in the art, any one of these methods may be used for the purposes of the present invention.

The term “Kd”, “koff” or “kdis” refers to the off rate constant of a particular interaction between a binding molecule and antigen. The off rate constant koff can be measured using bio-layer interferometry, for example, using the Octet™ system.

The term “Ka”, “kon” or “on-rate” refers to the association rate constant.

The term “R²” refers to the coefficient of determination.

The term “Response” refers to an antibody-antigen binding signal.

The term “in vitro” refers to a biological entity, a biological process, or a biological reaction outside the body under artificial conditions. For example, a cell grown in vitro is to be understood as a cell grown in an environment outside the body, e.g. in a test tube, a culture vial, or a microtiter plate.

The term “ED₅₀” (EC₅₀) (50% effective dose/concentration) refers to concentrations of a formulation producing 50% biological effect (which may include cytoxicity).

“Kabat numbering scheme” or “numbering according to Kabat” as used in the present application refers to the system for numbering of amino acid residues that are more variable (i.e. hypervariable) than other amino acid residues in variable regions of heavy and light chains of antibody (Kabat et al. Ann. N.Y. Acad. Sci., 190:382-93 (1971); Kabat et al. Sequences of Proteins of Immunological Interest, Fifth Edition, U.S. Department of Health and Human Services, NIH Publication No. 91-3242 (1991)).

As used in the present description and claims that follow, unless otherwise dictated by the context, the words “include” and “comprise”, or variations thereof such as “includes”, “including”, “comprises”, or “comprising”, will be understood to imply the inclusion of a stated integer or group of integers but not the exclusion of any other integer or group of integers.

### Antibody

The present invention relates to a monoclonal antibody or antigen-binding fragment thereof that specifically binds to AXL.

The term “monoclonal antibody” or “mAb” refers to an antibody that is synthesized and isolated as an individual clonal population of cells.

The antibody of the invention is a recombinant antibody.

The term “recombinant antibody” refers to an antibody that is expressed in a cell or cell line comprising nucleotide sequence(s) encoding an antibody, wherein said nucleotide sequence(s) is (are) not associated with the cell in nature.

In one aspect, the present invention relates to an monoclonal antibody or antigen-binding fragment thereof that specifically binds to AXL, comprising:
(a) a light chain variable domain comprising:
CDR1 with the amino acid sequence of SEQ ID NO: 1,
CDR2 with the amino acid sequence of SEQ ID NO: 2 and
CDR3 with the amino acid sequence of SEQ ID NO: 3; and
(b) a heavy chain variable domain comprising:
CDR1 with the amino acid sequence of SEQ ID NO: 7,
CDR2 with the amino acid sequence of SEQ ID NO: 8 and
CDR3 with the amino acid sequence of SEQ ID NO: 9.

Monoclonal antibodies according to the invention specifically bind to AXL fragments, comprising a distinct Ig-like C2-type 2 domain of AXL (AXL-Ig2) or two Ig-like C2-type 1 and Ig-like C2-type 1 domains (AXL-Ig1-Ig2), and to complete extracellular portion of AXL (ExcAXL) but do not bind to AXL fragments that comprise a distinct Ig-like C2-type 1 domain (AXL-Ig1). The monoclonal antibodies to AXL according to the present invention inhibit AXL and GAS6 binding leading to the activation of AXL-mediated cellular signaling.

In one embodiment of the invention, the antibody according to the invention is an isolated antibody.

The term “isolated” used to describe various antibodies according to the present description refers to an antibody which has been identified and isolated and/or regenerated from a cell or cell culture, in which the antibody is expressed. Impurities (contaminant components) from natural environment are materials which typically interfere with diagnostic or therapeutic uses of the polypeptide, and may include enzymes, hormones, and other proteinaceous or nonproteinaceous solutes. The isolated polypeptide is typically prepared by at least one purification step.

The term “antibody” or “immunoglobulin” (Ig) as used in the present description includes whole antibodies. The term “antibody” refers to a glycoprotein comprising at least two heavy (H) chains and two light (L) chains interconnected by disulfide bonds. Each heavy chain comprises a heavy chain variable region (abbreviated referred to in the present description as VH) and a heavy chain constant region. Each light chain consists of a light chain variable region (abbreviated referred to in the present description as VL) and light chain constant region. Preferably the light chain is a kappa (κ) light chain, and the constant domain CL is preferably C kappa (κ).

Antibodies according to the invention can be of any class (e.g., IgA, IgD, IgE, IgG, and IgM, preferably IgG), or subclass (e.g., IgG1, IgG2, IgG3, IgG4, IgAQ1 and IgA2, preferably IgG1).

VL and VH regions can be further subdivided into hyper-variability regions called complementarity determining regions (CDRs), interspersed between regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. The variable regions of heavy and light chains contain a binding domain that interacts with an antigen.

The constant regions of antibodies may mediate the binding of immunoglobulin to host tissues or factors, including various cells of the immune system (e.g. effector cells) and the first component (C1q) of the classical complement system.

The term “antigen-binding portion” of antibody or “antigen-binding fragment”, as used in the present description, refers to one or more antibody fragments that retain the ability to specifically bind to an antigen. It has been shown that the antigen-binding function of antibody can be performed by fragments of a full-length antibody. Examples of binding fragments which are included within the term “antigen-binding portion” of an antibody include (i) Fab-fragment, monovalent fragment, consisting of VL, VH, CL and CH1 domains; (ii) F(ab′)2 fragment, a bivalent fragment comprising two Fab-fragments linked by a disulfide bridge at the hinge region; (iii) Fd-fragment consisting of VH and CH1 domains; (iv) Fv-fragment consisting of VL and VH domains of a single arm of an antibody; (v) dAb-fragment (Ward et al., (1989) Nature 341:544-546), which consists of a VH/VHH domain. In addition, two regions of the Fv-fragment, VL and VH, are encoded by different genes, they can be joined using recombinant methods using a synthetic linker that enables to receive them as a single protein chain in which the VL and VH regions are paired to form monovalent molecules (known as a single-chai (scFv); see e.g. Bird et al. (1988) Science 242:423-426; and Huston et al. (1988) Proc. Natl. Acad. Sci. USA 85:5879-5883). It is assumed that such single-stranded molecules are also included within the term “antigen-binding portion” of antibody. Such antibody fragments are produced using conventional techniques known to those skilled in the art, and these fragments are screened in the same manner as intact antibodies are.

The antibody of the present invention “which specifically binds” a target antigen refers to an antibody that binds an antigen with sufficient affinity such that the antibody can be used as a diagnostic and/or therapeutic agent targeting a protein or cell or tissue expressing the antigen, and slightly cross-reacts with other proteins.

The term “specifically binds to” a particular polypeptide or an epitope on a particular target polypeptide may be described by example of a molecule having a Kd for the target of at least about 200 nM, or at least about 150 nM, or at least about 100 nM, or at least about 60 nM, or at least about 50 nM, or at least about 40 nM, or at least about 30 nM, or at least about 20 nM, or at least about 10 nM, or at least about 8 nM, or at least about 6 nM, or at least about 4 nM, or at least about 2 nM, or at least about 1 nM, or lower.

In one embodiment, the term “specific binding” refers to binding where a molecule binds to a particular polypeptide or epitope on a particular polypeptide without substantially binding to any other polypeptide or epitope on a polypeptide.

In some embodiments of the invention, the monoclonal antibody or antigen-binding fragment thereof comprises a light chain variable domain comprising the amino acid sequence of SEQ ID NO: 13.

In some embodiments of the invention, the monoclonal antibody or antigen-binding fragment thereof comprises a heavy chain variable domain comprising the amino acid sequence of SEQ ID NO: 14.

In some embodiments of the invention, the monoclonal antibody or antigen-binding fragment thereof includes:
(a) a light chain variable domain that comprises the amino acid sequence of SEQ ID NO: 13; and
(b) a heavy chain variable domain that comprises the amino acid sequence of SEQ ID NO: 14.

In some embodiments of the invention, the monoclonal antibody that specifically binds to AXL is a full-length IgG antibody.

In some embodiments of the invention, the monoclonal antibody that specifically binds to AXL is a full-length IgG antibody that is of human IgG1, IgG2, IgG3 or IgG4 isotype.

In some embodiments of the invention, the monoclonal antibody that specifically binds to AXL is a full-length IgG antibody that is of human IgG1 isotype.

In some embodiments of the invention, the monoclonal antibody comprises mutations in the Fc fragment, leading to increased ADCC, CDC and/or ADCP properties in the antibody.

In some embodiments of the invention, the monoclonal antibody comprises, in the Fc fragment, mutations S239D and I332E, according to the EU numbering scheme of amino acids of antibodies (Edelman G. M. et al., Proc. Natl. Acad. Sci. USA 63 (1969) pp. 78-85; Kabat, E. A., et al., Sequences of Proteins of Immunological Interest, 5th ed., Public Health Service, National Institutes of Health, Bethesda, MD, (1991).

In some embodiments of the invention, the monoclonal antibody comprises deletions 446G and 447K in the Fc fragment, according to the EU numbering scheme of amino acids of antibodies, in the CH3 region.

In some embodiments of the invention, the monoclonal antibody comprises a light chain comprising the amino acid sequence of SEQ ID NO: 15.

In some embodiments of the invention, the monoclonal antibody comprises a heavy chain comprising an amino acid sequence that is selected from the group: SEQ ID NO: 16 or SEQ ID NO: 17.

In some embodiments of the invention, the monoclonal antibody includes:
(i) (a) a light chain comprising the amino acid sequence of SEQ ID NO: 15, and
(b) a heavy chain comprising the amino acid sequence of SEQ ID NO: 16;
or
(ii) (a) a light chain comprising the amino acid sequence of SEQ ID NO: 15, and
(b) a heavy chain comprising the amino acid sequence of SEQ ID NO: 17.

In some embodiments of the invention, the monoclonal antibody that specifically binds to AXL is an antibody that is selected from the group: 01_004 and 01_004_2.

In some embodiments of the invention, the monoclonal antibody that specifically binds to AXL is antibody 01_004.

Antibody 01_004 includes:
(a) a light chain comprising the amino acid sequence of SEQ ID NO: 15; and
(b) a heavy chain comprising the amino acid sequence of SEQ ID NO: 16.

Antibody 01_004 includes:
(a) a light chain variable domain that comprises the amino acid sequence of SEQ ID NO: 13; and
(b) a heavy chain variable domain that comprises the amino acid sequence of SEQ ID NO: 14.

Antibody 01_004 includes:
(a) a light chain variable domain comprising:
(i) CDR1 (Kabat) with the amino acid sequence of SEQ ID NO: 1,
(ii) CDR2 (Kabat) with the amino acid sequence of SEQ ID NO: 2 and
(iii) CDR3 (Kabat) with the amino acid sequence of SEQ ID NO: 3,
and
(b) a heavy chain variable domain comprising:
(i) CDR1 (Kabat) with the amino acid sequence of SEQ ID NO: 7,
(ii) CDR2 (Kabat) with the amino acid sequence of SEQ ID NO: 8 and
(iii) CDR3 (Kabat) with the amino acid sequence of SEQ ID NO: 9.

Antibody 01_004 includes:
(a) a light chain variable domain comprising:
(i) CDR1 (Chothia) with the amino acid sequence of SEQ ID NO: 4,
(ii) CDR2 (Chothia) with the amino acid se ice of SEQ ID NO: 5 and
(iii) CDR3 (Chothia) with the amino acid sequence of SEQ ID NO: 6,
and
(b) a heavy chain variable domain comprising:
(i) CDR1 (Chothia) with the amino acid sequence of SEQ ID NO: 10,
(ii) CDR2 (Chothia) with the amino acid sequence of SEQ ID NO: 11 and
(iii) CDR3 (Chothia) with the amino acid sequence of SEQ ID NO: 12.

In some embodiments of the invention, the monoclonal antibody that specifically binds to AXL is antibody 01_004_2.

Antibody 01_004_2 includes:
(a) a light chain comprising the amino acid sequence of SEQ ID NO: 15; and
(b) a heavy chain comprising the amino acid sequence of SEQ ID NO: 17.

Antibody 01_004_2 includes:
(a) a light chain variable domain that comprises the amino acid sequence of SEQ ID NO: 13;
and
(b) a heavy chain variable domain that comprises the amino acid sequence of SEQ ID NO: 14.

Antibody 01_004_2 includes:
(a) a light chain variable domain comprising:
(i) CDR1 (Kabat) with the amino acid sequence of SEQ ID NO: 1,
(ii) CDR2 (Kabat) with the amino acid sequence of SEQ ID NO: 2 and
(iii) CDR3 (Kabat) with the amino acid sequence of SEQ ID NO: 3,
and
(b) a heavy chain variable domain comprising:
(i) CDR1 (Kabat) with the amino acid sequence of SEQ ID NO: 7,
(ii) CDR2 (Kabat) with the amino acid sequence of SEQ ID NO: 8 and
(iii) CDR3 (Kabat) with the amino acid sequence of SEQ ID NO: 9.

Antibody 01_004_2 includes:
(a) a light chain variable domain comprising:
(i) CDR1 (Chothia) with the amino acid sequence of SEQ ID NO: 4,
(ii) CDR2 (Chothia) with the amino acid sequence of SEQ ID NO: 5 and
(iii) CDR3 (Chothia) with the amino acid sequence of SEQ ID NO: 6,
and
(b) a heavy chain variable domain comprising:
(i) CDR1 (Chothia) with the amino acid sequence of SEQ ID NO: 10,
(ii) CDR2 (Chothia) with the amino acid sequence of SEQ ID NO: 11 and
(iii) CDR3 (Chothia) with the amino acid sequence of SEQ ID NO: 12.

The hypervariable regions of variable domains of light and heavy chains (LCDR1, 2, 3 and HCDR1, 2, 3) of all the above antibodies are provided in accordance with the Kabat and Chothia nomenclature. Those skilled will appreciate that the hypervariable ions of variable domains of light and heavy chains (LCDR1, 2, 3 and HCDR1, 2, 3) may also be represented in accordance with other commonly known numbering scheme, for example, IMGT or AbM. Thus, all of the above antibodies which are characterized by means of hypervariable regions of variable domains of light and heavy chains (LCDR1, 2, 3 and HCDR1, 2, 3) using the IMGT or AbM numbering scheme are also encompassed by the present invention.

### Nucleic Acid Molecule

In one aspect, the present invention relates to a nucleic acid that encodes any one of the above antibody or antigen-binding fragment thereof that specifically binds to AXL.

In any one of said embodiments, the nucleic acid molecules may be isolated.

The terms “nucleic acid”, “nucleic sequence” or “nucleic acid sequence”, “polynucleotide”, “oligonucleotide”, “polynucleotide sequence” and “nucleotide sequence”, used interchangeably in the present description, mean a precise sequence of nucleotides, modified or not, determining a fragment or a region of a nucleic acid, containing unnatural nucleotides or not, and being either a double-strand DNA or RNA, a single-strand DNA or RNA, or transcription products of said DNAs.

Unless otherwise indicated, the term nucleotide sequence encompasses its complement. Thus, a nucleic acid having a particular sequence should be understood as one which encompasses the complementary strand thereof with the complementary sequence thereof.

An “isolated” nucleic acid molecule is one which is identified and separated from at least one nucleic acid molecule-impurity. An isolated nucleic acid molecule is different from the form or set in which it is found under natural conditions. Thus, an isolated nucleic acid molecule is different from a nucleic acid molecule that exists in cells under natural conditions.

In one aspect, the present invention relates to a nucleic acid molecule comprising a nucleotide sequence that encodes an amino acid sequence selected from SEQ ID NO: 18-22. A nucleic acid molecule may also comprise any combination of said nucleotide sequences.

As would be appreciated by those skilled in the art, because of the redundancy of the genetic code, a variety of different DNA sequences can encode the amino acid sequence of the light chain or heavy chain of the antibody according to the invention or fragments thereof (VH, VL, CDR, etc.). It is well within the skill of those trained in the art to create these alternative DNA sequences encoding one and the same amino acid sequences. Such variant DNA sequences are within the scope of the present invention.

In some embodiments of the invention, the nucleic acid is DNA.

The nucleic acid molecule of the invention may be isolated from any source that produces the monoclonal antibody or antigen-binding fragment thereof that specifically binds to AXL. In certain embodiments of the invention, the nucleic acid molecule of the invention may be synthesized by way of chemical synthesis, rather than isolated.

In some embodiments of the invention, the nucleic acid is a nucleic acid that encodes the amino acid sequence of the light chain variable domain of antibodies 01_004 and 01_004_2, and includes the nucleotide sequence with SEQ ID NO: 18.

In some embodiments of the invention, the nucleic acid is a nucleic acid that encodes the amino acid sequence of the heavy chain variable domain of antibodies 01_004 and 01_004_2, and includes the nucleotide sequence with SEQ ID NO: 19.

In some embodiments of the invention, the nucleic acid is a nucleic acid that encodes the amino acid sequence of the light chain of antibodies 01_004 and 01_004_2, and includes the nucleotide sequence with SEQ ID NO: 20.

In some embodiments of the invention, the nucleic acid is a nucleic acid that encodes the amino acid sequence of the heavy chain of antibody 01_004, and includes the nucleotide sequence with SEQ ID NO: 21.

In some embodiments of the invention, the nucleic acid is a nucleic acid that encodes the amino acid sequence of the heavy chain of antibody 01_004_2, and includes the nucleotide sequence with SEQ ID NO: 22.

The nucleic acid molecules may be used to express the monoclonal antibody or antigen-binding fragment thereof that specifically binds to AXL.

### Vector

In one aspect, the present invention relates to an expression vector comprising any one of the above nucleic acid molecules that encode the corresponding amino acid sequences of the antibody that specifically binds to AXL, or portions thereof (for example, heavy chain and/or light chain binding domain sequences). The present invention relates to a vector suitable for the expression of any one of nucleotide sequences described herein.

The term “vector” as used herein means a nucleic acid molecule capable of transporting other nucleic acid to which it has been linked.

As used in the present description, the term “expression” is defined as the transcription and/or translation of a particular nucleotide sequence driven by its promoter.

In some embodiments of the invention, the vector is a plasmid, i.e. a circular double stranded piece of DNA into which additional DNA segments may be inserted.

In some embodiments of the invention, the vector is a viral (expression) vector, wherein additional DNA segments may be inserted into the viral genome.

In some embodiments of the invention, the vectors are capable of autonomous replication in a host cell into which they have been introduced (e.g. bacterial vectors having a bacterial site of replication origin and episomal vectors). In further embodiments of the invention, vectors (e.g. non-episomal vectors) may be integrated into the genome of a host cell upon introduction into a host cell, and thereby are replicated along with the host gene. Moreover, certain vectors are capable of directing the expression of genes to which they are operably linked. Such vectors are referred to herein as “recombinant expression vectors” (or simply, “expression vectors”).

In some embodiments of the invention, expression vectors include plasmids, retroviruses, adenoviruses, adeno-associated viruses (AAVs), plant viruses, such as cauliflower mosaic virus, tobacco mosaic virus, cosmids, YACs, and the like. DNA molecules may be inserted into a vector such that transcriptional and translational control sequences within the vector serve their intended function of regulating the transcription and translation of DNA. An expression vector and expression control sequences may be chosen to be compatible with the expression host cell used.

In one embodiment of the invention, DNA molecules encoding partially or fully heavy and light chain sequences can be inserted into distinct vectors.

In one embodiment, any combination of the above DNA molecules is introduced into the same expression vector.

In one embodiment of the invention, DNA molecules may be introduced into an expression vector by standard methods (e.g. ligation of complementary restriction sites on a gene fragment of antibody and vector, or blunt end ligation if no restriction sites are present).

In some embodiments of the invention, a suitable vector is one that includes restriction sites such that any VH or VL sequence can easily be inserted and expressed, as described above. A recombinant expression vector can also encode a signal peptide that facilitates secretion of an antibody chain from a host cell. An antibody chain gene may be cloned into a vector such that the signal peptide is linked in-frame to the amino terminus of an immunoglobulin chain. A signal peptide may be an immunoglobulin signal peptide or a heterologous signal peptide (i.e. a signal peptide from a non-immunoglobulin protein).

In some embodiments of the invention, the vector may include an expression control sequence. The term “expression control sequence” as used in the present description refers to polynucleotide sequences that are necessary to effect the expression and processing of coding sequences to which they are inserted. It will be understood by those skilled in the art that the design of an expression vector, including the selection of expression control sequences, may depend on such factors as the choice of the type of a host cell to be transformed, the required level of expression of antibody, and so forth. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such expression control sequences differs depending upon the host organism; in prokaryotes, such expression control sequences typically include a promoter, a ribosome binding site, as well as transcription termination sequences; in eukaryotes, such expression control sequences typically include promoters and transcription termination sequences. Preferred expression control sequences for an expression host cell in a mammal include viral elements that ensure high levels of protein expression in mammalian cells, such as promoters and/or enhancers derived from a retroviral LTR, cytomegalovirus (CMV) (such as a CMV promoter/enhancer), simian virus 40 (SV40) (such as a SV40 promoter/enhancer), adenovirus, (e.g. the major late promoter adenovirus (AdMLP)), polyomavirus and strong mammalian promoters such as TTR promoter, native immunoglobulin promoter or actin promoter. Expression control sequences encompass at least all components whose presence is important for expression and processing.

In some embodiments of the invention, in addition to antibody chain genes and expression control sequences, the recombinant expression vectors of invention may carry additional sequences, such as sequences that regulate replication of a vector in host cells (e.g. origins of replication) and selectable marker genes. The selectable marker gene facilitates the selection of host cells into which a vector has been introduced.

### Host Cell

In one aspect, the present invention relates to a method for producing a host cell to produce any above antibody or antigen-binding fragment thereof that specifically binds to AXL, and includes transformation of the cell with the above vector.

In one aspect, the present invention relates to a host cell for producing any above antibody or antigen-binding fragment thereof that specifically binds to AXL, comprising any one of the above nucleic acids.

The term “host cell” as used herein refers to a cell into which a recombinant expression vector has been introduced. The present invention relates to host cells, which may include, for example, the above-described vector according to the invention. The present invention further relates to host cells that comprise, for example, a nucleotide sequence encoding a heavy chain or antigen-binding portions thereof, a nucleotide sequence encoding a light chain or antigen-binding portions thereof, or both. It should be understood that “host cell” refers not only to a particular subject cell but to the progeny of such cell as well. Since modifications may occur in succeeding generations due to either mutation or environmental influences, such progeny may not, in fact, be identical to a parental cell; however, such cells are still included within the scope of the term “host cell” as used herein.

Nucleic acid molecules encoding the monoclonal antibody or antigen-binding fragment thereof that specifically binds to AXL according to the invention and vectors comprising these nucleic acid molecules may be used for transfection of a mammalian cell, plant cell, bacterial cell, or yeast cell. Transfection may be carried out by any known method for introducing polynucleotides into a host cell. Methods for introducing heterologous polynucleotides into mammalian cells are well known in the art and include dextran-mediated transfection, cationic polymer-nucleic acid complex transfection, calcium phosphate precipitation, polybrene-mediated transfection, protoplast fusion, encapsulation of the polynucleotides in liposomes, and direct microinjection of DNA into nuclei. In addition, the nucleic acid molecules may be introduced into mammalian cells by viral (expression) vectors.

Mammalian cell lines used as hosts for transformation are well known in the art and include a plurality of immortalized cell lines available. These include, e.g., Chinese hamster ovary (CHO) cells, NS0 cells, SP2 cells, HEK-293T cells, FreeStyle 293 cells (Invitrogen), NIH-3T3 cells, HeLa cells, baby hamster kidney (BHK) cells, African green monkey kidney cells (COS), human hepatocellular carcinoma cells (e.g., Hep G2), A549, SK-HEP1, HUH7, Hep-RG cells and a number of other cell lines. Cell lines are selected by way of determining which cell lines have high expression levels and provide for necessary characteristics of the protein being produced. Other cell lines that may be used are insect cell lines, such as Sf9 or Sf21 cells. When recombinant expression vectors encoding the monoclonal antibody or antigen-binding fragment thereof that specifically binds to AXL are introduced into mammalian host cells, the antibodies or fragments thereof are produced by culturing the host cells for a period of time sufficient to allow for expression of the antibodies or fragments thereof in host cells or, more preferably, secretion of the antibodies or fragments thereof into the culture medium in which the host cells are grown. The monoclonal antibody or antigen-binding fragment thereof that specifically binds to AXL may be isolated from culture medium using standard protein purification techniques. Plant host cells include e.g. Nicotiana, Arabidopsis, duckweed, corn, wheat, potato, etc. Bacterial host cells include Escherichia and Streptomyces species. Yeast host cells include Schizosaccharomyces pombe, Saccharomyces cerevisiae and Pichia pastoris.

Furthermore, level of production of the monoclonal antibody or antigen-binding fragment thereof that specifically binds to AXL from a production cell line may be enhanced using a number of known techniques. For example, the glutamine synthetase gene expression system (the GS system) is a common approach for enhancing expression under certain conditions.

It is likely that the monoclonal antibody or antigen-binding fragment thereof that specifically binds to AXL from various cell lines will have a different glycosylation profile as compared to one another. However, the monoclonal antibody or antigen-binding fragment thereof that specifically binds to AXL encoded by nucleic acid molecules described herein, or comprising amino acid sequences provided herein are part of the present invention, regardless of the glycosylation of the binding molecules, and, in general, regardless of the presence or absence of post-translational modifications.

The above host cell does not relate to a host cell produced using human embryos.

The above host cell does not relate to a host cell produced by modifying the genetic integrity of human germline cells.

### Method for Producing Antibody

In one aspect, the present invention relates to a method for producing the antibody or antigen-binding fragment thereof that specifically binds to AXL, comprising culturing the above host cell in a growth medium under conditions sufficient to produce said antibody or antigen-binding fragment thereof, followed by isolation and purification of the resulting antibody or antigen-binding fragment thereof.

### Pharmaceutical Compositions

Another aspect of the invention is a pharmaceutical composition comprising, as an active ingredient (or as the only active ingredient), the monoclonal antibody according to the present invention or antigen-binding fragment thereof that specifically binds to AXL.

In one aspect, the present invention relates to a pharmaceutical composition that comprises any above-mentioned antibody or antigen-binding fragment thereof in combination with one or more pharmaceutically acceptable excipients.

In one aspect, the present invention relates to a pharmaceutical composition used for treating an AXL-mediated disease or disorder, which comprises any above antibody or antigen-binding fragment thereof in combination with one or more pharmaceutically acceptable excipients.

In one aspect, the present invention relates to a pharmaceutical composition used for treating an AXL-mediated disease or disorder, which comprises any above antibody or antigen-binding fragment thereof in a therapeutically effective amount in combination with one or more pharmaceutically acceptable excipients.

“Pharmaceutical composition” means a composition comprising the antibody according to the invention and at least one of components selected from the group consisting of pharmaceutically acceptable and pharmacologically compatible fillers, solvents, diluents, carriers, auxiliary, distributing and sensing agents, delivery agents.

The term “pharmaceutically acceptable” refers to one or more compatible liquid or solid components that are suitable for administration in a mammal, preferably in a human.

The term “excipient” is used herein to describe any ingredient other than the antibody according to the present invention. These are substances of inorganic or organic nature which are used in the pharmaceutical production/manufacturing in order to give drug products the necessary physicochemical properties.

In some embodiments, the compositions are intended to improve, prevent, or treat diseases or disorders that may be mediated by AXL.

The term “AXL-mediated disease or disorder” refers to any disease or disorder that is either directly, or indirectly associated with AXL, including etiology, development, progression, persistence or pathology of a disease or disorder.

“Treat”, “treatment” and “therapy” refer to a method of alleviating or abrogating a biological disorder and/or at least one of attendant symptoms thereof. Further, references herein to “treatment” include references to curative, palliative and prophylactic treatment.

The term “disorder” means any condition that would benefit from treatment according to the present invention. The definition of the term includes chronic and acute disorders or diseases including those pathological conditions that predispose the mammal to the disorder in question.

“Therapeutically effective amount” refers to that amount of the therapeutic agent being administered during treatment which will relieve to some extent one or more of the symptoms of the disease being treated. A therapeutically effective amount may vary according to factors such as the particular condition being treated, the age, sex and weight of the patient, and whether the monoclonal antibody or antigen-binding fragment thereof that specifically binds to AXL is being administered as a stand-alone treatment or in combination with one or more additional drugs or treatments.

In one aspect, the subject of treatment, or patient, is a mammal, preferably a human subject. Said subject may be either male or female, of any age.

The pharmaceutical compositions of the present invention and methods of preparation thereof will be undoubtedly apparent to those skilled in the art. The pharmaceutical compositions should preferably be manufactured in compliance with the GMP (Good Manufacturing Practice) requirements.

In some embodiments of the pharmaceutical composition, it may include a buffer composition, tonicity agents (osmolyte or osmotic agent), stabilizers and/or solubilizers.

The pharmaceutical composition according to the invention is a stable composition.

A pharmaceutical composition is “stable” if the active agent retains physical stability and/or chemical stability and/or biological activity thereof during the specified shelf life at storage temperature, for example, of 2-8° C. Preferably, the active agent ins both physical and chemical stability, as well as biological activity. Storage period is adjusted based on the results of stability test in accelerated or natural aging conditions.

The pharmaceutical composition according to the present invention is suitable for parenteral administration as sterile formulations intended for administration in a subject body through the breach in skin or mucosal barriers, bypassing the gastrointestinal tract by virtue of injection, infusion and implantation. In particular, it is contemplated that parenteral administration includes, inter alia, subcutaneous, intraperitoneal, intramuscular, intravenous, intraarterial, intrathecal, intraventricular, intraurethral, intracranial, intrasynovial, transdermal injection or infusion; and kidney dialytic infusion techniques. Intra-tumor delivery, for example, intra-tumor injection, may also be employed. Regional perfusion is also contemplated.

In some embodiments, the pharmaceutical composition is administered intravenously.

In some embodiments, intravenous administration is carried out by using infusion, prolonged infusion, or long-lasting continuous infusion.

In some embodiments, the pharmaceutical composition is administered subcutaneously.

In some embodiments, subcutaneous administration is carried out by using subcutaneous injection.

In some embodiments, the pharmaceutical composition is an injectable dosage form.

In some embodiments, the pharmaceutical composition is a solution for intravenous administration.

In some embodiments, the injectable dosage form is an infusion solution.

In some embodiments, the injectable dosage form is a solution for subcutaneous administration.

Injectable formulations may be manufactured without limitation, in unit dosage form, such as in ampoules, vials, plastic containers, pre-filled syringes, autoinjection devices.

In some embodiments, the pharmaceutical composition is a pharmaceutical composition provided in dry, i.e. powder or granular, form for reconstitution with a suitable solvent (e.g., sterile pyrogen-free water) prior to administration. Such medicinal formulation may be prepared by, for example, lyophilization, i.e. a process, which is known in the art as freeze drying, and which involves freezing a product followed by removal of solvent from frozen material.

In some embodiments, the pharmaceutical composition is a lyophilizate for preparing a solution for infusion.

In some embodiments, the pharmaceutical composition is a lyophilizate for preparing a solution for subcutaneous administration.

In some embodiments, the pharmaceutical composition is a concentrate for preparing a solution for infusion.

In some embodiments, the pharmaceutical composition is a concentrate for preparing a solution for subcutaneous administration.

In one aspect, the present invention relates to a pharmaceutical composition that comprises a monoclonal antibody according to the present invention or antigen-binding fragment thereof that specifically binds to AXL and at least one other therapeutically active compound.

In one aspect, the present invention relates to a pharmaceutical composition for treating an AXL-mediated disease or disorder, comprising any above antibody or antigen-binding fragment thereof and at least one other therapeutically active compound.

In one aspect, the present invention relates to a pharmaceutical composition comprising any above antibody or antigen-binding fragment thereof and further at least one other therapeutically active compound.

In one aspect, the present invention relates to a pharmaceutical composition for treating an AXL-mediated disease or disorder, comprising any above antibody or antigen-binding fragment thereof and further at least one other therapeutically active compound.

In one aspect, the present invention relates to a pharmaceutical composition for treating an AXL-mediated disease or disorder, comprising any above antibody or antigen-binding fragment thereof and at least one other therapeutically active compound, which is an antibody, a small molecule, a hormone therapy agent or a combination thereof.

In some embodiments of the pharmaceutical composition, the other therapeutically active compound is an immune checkpoint inhibitor.

The term “immune checkpoint inhibitor” (or “checkpoint inhibitor”) refers to compounds that inhibit the activity of immune checkpoints. Inhibition includes reduction of function and full blockade. Examples of inhibitory checkpoint molecules include B7-H3, B7-H4, BTLA, CTLA-4, KIR, PD-1, PD-L1, PD-L2, LAG-3, TIM-3, TIGIT, and VISTA. In some embodiments of the invention, the immune checkpoint inhibitor is an antibody that specifically recognizes an immune checkpoint protein. A number of immune checkpoint inhibitors are known and in analogy of these known immune checkpoint protein inhibitors, alternative immune checkpoint inhibitors may be developed in the near future. The immune checkpoint inhibitors include, but are not limited to, peptides, antibodies, nucleic acid molecules, and low molecular weight compounds.

In some embodiments of the pharmaceutical composition, the immune checkpoint inhibitor is selected from a PD-1 inhibitor, PD-L1 inhibitor, or CTLA-4 inhibitor.

In some embodiments of the pharmaceutical composition, the PD-1 inhibitor is an antibody that specifically binds to PD-1.

In some embodiments of the pharmaceutical composition, the antibody that specifically binds to PD-1 is selected from the group: prolgolimab, pembrolizumab, nivolumab.

In some embodiments of the pharmaceutical composition, the CTLA-4 inhibitor is an antibody that specifically binds to CTLA-4.

In some embodiments of the pharmaceutical composition, the antibody that specifically binds to CTLA-4 is ipilimumab or nurulimab.

In some embodiments of the pharmaceutical composition, the PD-L1 inhibitor is an antibody that specifically binds to PD-L1.

In some embodiments of the pharmaceutical composition, the antibody that specifically binds to PD-L1 is selected from the group: durvalumab, avelumab, atezolizumab, manelimab.

In some embodiments of the pharmaceutical composition, the other therapeutically active compound is selected from the group: EGFR-TKI, afatinib, erlotinib, gefitinib, osimertinib, doxorubicin, paclitaxel, capecitabine, carboplatin, cisplatin, lapatinib, trastuzumab emtansine, docetaxel, cyclophosphamide, topotecan, niraprib, olaparib, pembrolizumab, platinum agents, bevacizumab, prolgolimab, nivolumab, vemurafenib, dabrafenib, cobimetinib, trametinib, etoposide, temozolomide, lomustine, imatinib, dasatinib, vincristine, doxorubicin derivatives, cytarabine, fludarabine, rituximab, venetoclax, PARP inhibitors, irinotecan, trastuzumab, gemcitabine, atezolizumab, fluoropyrimidine, cetuximab, ipilimumab, methotrexate, ifosfamide, docorubicin, BRAF inhibitors, MEK inhibitors, donepezil, rivastigmine, galantamine, nintedanib, etiotropic therapy, anti-inflammatory therapy, glucocorticoid, antithrombotic therapy or any combination thereof.

In some embodiments of the pharmaceutical composition, the AXL-mediated disease or disorder is selected from the group: non-small cell lung cancer, non-small cell lung cancer with a EGFR mutation, breast carcinoma, HER2-positive breast cancer, four time-negative breast cancer, triple-negative breast cancer (TNBC), ovarian cancer, platinum-resistant ovarian cancer, prostate cancer, docetaxel-resistant prostate cancer, endometrial cancer and uterine sarcoma, endometrioid adenocarcinoma, uterine serous carcinoma, skin melanoma, neuroblastoma, glioblastoma, head and neck squamous cell carcinoma, stomach cancer, renal cell carcinoma, urothelial carcinoma, colorectal cancer, colon cancer, hepatocellular carcinoma, pancreatic cancer, biliary cancer, malignant neoplasm of the extrahepatic bile duct, intrahepatic bile duct cancer, malignant neoplasms of the gallbladder, osteosarcoma, Ewing sarcoma, neoplasms with high-level AXL expression, chronic myeloid leukemia, chronic lymphocytic leukemia, acute myeloid leukemia, acute lymphoblastic leukemia, chronic liver disease, non-alcoholic steatohepatitis, Alzheimer's disease or idiopathic pulmonary fibrosis.

Therapeutic Use of Monoclonal Antibody or Antigen-Binding Fragment Thereof that Specifically Binds to AXL

In one aspect, the antibody or antigen-binding fragment thereof that specifically binds to AXL is used in the treatment of disorders mediated by AXL activity.

In one aspect, the subject of treatment, or patient, is a mammal, preferably a human subject. Said subject may be either male or female, of any age.

In one aspect, the present invention relates to a method for treating an AXL-mediated disease or disorder, comprising administering in a subject in need of such treatment any above antibody or antigen-binding fragment thereof or said pharmaceutical composition, in a therapeutically effective amount.

In one aspect, the present invention relates to a method for treating an AXL-mediated disease or disorder, comprising administering in a subject in need of such treatment any above antibody or antigen-binding fragment thereof and at least one other therapeutically active compound in a therapeutically effective amount.

In some embodiments of the method of treatment, the AXL-mediated disease or disorder is selected from the group: non-small cell lung cancer, non-small cell lung cancer with a EGFR mutation, breast carcinoma, HER2-positive breast cancer, four time-negative breast cancer, triple-negative breast cancer (TNBC), ovarian cancer, platinum-resistant ovarian cancer, prostate cancer, docetaxel-resistant prostate cancer, endometrial cancer and uterine sarcoma, endometrioid adenocarcinoma, uterine serous carcinoma, skin melanoma, neuroblastoma, glioblastoma, head and neck squamous cell carcinoma, stomach cancer, renal cell carcinoma, urothelial carcinoma, colorectal cancer, colon cancer, hepatocellular carcinoma, pancreatic cancer, biliary cancer, malignant neoplasm of the extrahepatic bile duct, intrahepatic bile duct cancer, malignant neoplasms of the gallbladder, osteosarcoma, Ewing sarcoma, neoplasms with high-level AXL expression, chronic myeloid leukemia, chronic lymphocytic leukemia, acute myeloid leukemia, acute lymphoblastic leukemia, chronic liver disease, non-alcoholic steatohepatitis, Alzheimer's disease or idiopathic pulmonary fibrosis.

In some embodiments of the method for treatment, the other therapeutically active compound is an antibody, small molecule, hormone therapy agent, or a combination thereof.

In some embodiments of the method of treatment, the other therapeutically active compound is an immune checkpoint inhibitor.

In some embodiments of the method of treatment, the immune checkpoint inhibitor is selected from a PD-1 inhibitor, PD-L1 inhibitor, or CTLA-4 inhibitor.

In some embodiments of the method of treatment, the PD-1 inhibitor is an antibody that specifically binds to PD-1.

In some embodiments of the invention, the PD-1 inhibitor is an antibody that specifically binds to PD-1. Examples of antibodies that specifically bind to PD-1 include pembrolizumab, nivolumab, prolgolimab, toripalimab, cemiplimab, sintilimab and others. The most preferred ones are prolgolimab, pembrolizumab, nivolumab.

In some embodiments of the method of treatment, the antibody that specifically binds to PD-1 is selected from the group comprising prolgolimab, pembrolizumab, nivolumab.

In some embodiments of the method of treatment, the CTLA-4 inhibitor is an antibody that specifically binds to CTLA-4.

In some embodiments of the invention, the CTLA-4 inhibitor is an antibody that specifically binds to CTLA-4. Examples of antibodies that specifically bind to CTLA4 include ipilimumab, tremelimumab, zalifrelimab, nurulimab and others. The most preferred ones are ipilimumab or nurulimab.

In some embodiments of the method of treatment, the antibody that specifically binds to CTLA-4 is ipilimumab or nurulimab.

In some embodiments of the method of treatment, the PD-L1 inhibitor is an antibody that specifically binds to PD-L1.

In some embodiments of the method of treatment, the antibody that specifically binds to PD-L1 is selected from the group: durvalumab, avelumab, atezolizumab, manelimab.

In one aspect, the present invention relates to the use of the above antibody or antigen-binding fragment thereof or the above pharmaceutical composition for treating in a subject in need of such treatment an AXL-mediated disease or disorder.

In one aspect, the present invention relates to the use of the above antibody or antigen-binding fragment thereof and at least one other therapeutically active compound for treating in a subject in need of such treatment an AXL-mediated disease or disorder.

In some embodiments of the use, the AXL-mediated disease or disorder is selected from the group: non-small cell lung cancer, non-small cell lung cancer with a EGFR mutation, breast carcinoma, HER2-positive breast cancer, four time-negative breast cancer, triple-negative breast cancer (TNBC), ovarian cancer, platinum-resistant ovarian cancer, prostate cancer, docetaxel-resistant prostate cancer, endometrial cancer and uterine sarcoma, endometrioid adenocarcinoma, uterine serous carcinoma, skin melanoma, neuroblastoma, glioblastoma, head and neck squamous cell carcinoma, stomach cancer, renal cell carcinoma, urothelial carcinoma, colorectal cancer, colon cancer, hepatocellular carcinoma, pancreatic cancer, biliary cancer, malignant neoplasm of the extrahepatic bile duct, intrahepatic bile duct cancer, malignant neoplasms of the gallbladder, osteosarcoma, Ewing sarcoma, neoplasms with high-level AXL expression, chronic myeloid leukemia, chronic lymphocytic leukemia, acute myeloid leukemia, acute lymphoblastic leukemia, chronic liver disease, non-alcoholic steatohepatitis, Alzheimer's disease or idiopathic pulmonary fibrosis.

In some embodiments of the use, the other therapeutically active compound is an antibody, small molecule, hormone therapy agent, or a combination thereof.

In some embodiments of the use, the other therapeutically active compound is an immune checkpoint inhibitor.

In some embodiments of the use, the immune checkpoint inhibitor is selected from a PD-1 inhibitor, PD-L1 inhibitor, or CTLA-4 inhibitor.

In some embodiments of the use, the PD-1 inhibitor is an antibody that specifically binds to PD-1.

In some embodiments of the use, the antibody that specifically binds to PD-1 is selected from the group: prolgolimab, pembrolizumab, nivolumab.

In some embodiments of the use, the CTLA-4 inhibitor is an antibody that specifically binds to CTLA-4.

In some embodiments of the use, the antibody that specifically binds to CTLA-4 is ipilimumab or nurulimab.

In some embodiments of the use, the PD-L1 inhibitor is an antibody that specifically binds to PD-L1.

In some embodiments of the use, the antibody that specifically binds to PD-L1 is selected from the group: durvalumab, avelumab, atezolizumab, manelimab.

The uses or methods used herein relating to the antibody or antigen-binding fragment thereof that specifically binds to AXL with one or more other therapeutic agents are contemplated to mean, refer to and include the following:
1) simultaneous administration of such combination of the antibody or antigen-binding fragment thereof that specifically binds to AXL and therapeutic agent to a patient in need of treatment, when such components are formulated together into a single dosage form which releases said components at substantially the same time to said patient,
2) simultaneous administration of such combination of the antibody or antigen-binding fragment thereof that specifically binds to AXL and therapeutic agent to a patient in need of treatment, when such components are formulated apart from each other into separate dosage forms which are taken at substantially the same time by said patient, whereupon said components are released at substantially the same time to said patient,
3) sequential administration of such combination of the antibody or antigen-binding fragment thereof that specifically binds to AXL and therapeutic agent to a patient in need of treatment, when such components are formulated apart from each other into separate dosage forms which are taken at consecutive times by said patient with a significant time interval between each administration, whereupon said components are released at substantially different times to said patient; and
4) sequential administration of such combination of the antibody or antigen-binding fragment thereof that specifically binds to AXL and therapeutic agent to a patient in need of treatment, when such components are formulated together into a single dosage form which releases said components in a controlled manner, whereupon they are concurrently, consecutively, or jointly released at the same and/or different times to said patient, where each portion may be administered by either the same or different routes.

The antibody or antigen-binding fragment thereof that specifically binds to AXL may be administered without further therapeutic treatment, i.e. as an independent therapy.

In some embodiments of the method of treatment or of use, the antibody or antigen-binding fragment thereof that specifically binds to AXL may be administered in combination with EGFR-TKI agents (afatinib, erlotinib, gefitinib and/or osimertinib) and/or an immune checkpoint inhibitor selected from a PD-1 inhibitor or PD-L1 inhibitor.

In some embodiments of the method of treatment or of use, the antibody or antigen-binding fragment thereof that specifically binds to AXL may be administered in combination with chemotherapy (doxorubicin, paclitaxel, capecitabine, carboplatin and/or cisplatin) and/or an immune checkpoint inhibitor selected from a PD-1 inhibitor or PD-L1 inhibitor.

In some embodiments of the method of treatment or of use, the antibody or antigen-binding fragment thereof that specifically binds to AXL may be administered in combination with targeted therapy (lapatinib) and/or cytostatic therapy, trastuzumab emtansine.

In some embodiments of the method of treatment or of use, the antibody or antigen-binding fragment thereof that specifically binds to AXL may be administered in combination with chemotherapy (docetaxel, paclitaxel, cyclophosphamide and/or topotecan) and/or targeted therapy (niraprib and/or olaparib).

In some embodiments of the method of treatment or of use, the antibody or antigen-binding fragment thereof that specifically binds to AXL may be administered in combination with chemotherapy (docetaxel, paclitaxel, cyclophosphamide and/or topotecan) and/or combined chemohormonotherapy and/or targeted therapy (niraprib and/or olaparib) and/or pembrolizumab.

In some embodiments of the method of treatment or of use, the antibody or antigen-binding fragment thereof that specifically binds to AXL may be administered in combination with chemotherapy (platinum agents, topotecan and/or paclitaxel) and/or targeted therapy (bevacizumab) and/or pembrolizumab.

In some embodiments of the method of treatment or of use, the antibody or antigen-binding fragment thereof that specifically binds to AXL may be administered in combination with an immune checkpoint inhibitor (prolgolimab, nivolumab or pembrolizumab) and/or targeted therapy (vemurafenib/dabrafenib and/or cobimetinib/trametinib).

In some embodiments of the method of treatment or of use, the antibody or antigen-binding fragment thereof that specifically binds to AXL may be administered in combination with cytotoxic chemotherapy (platinum agents, etoposide and/or temozolomide).

In some embodiments of the method of treatment or of use, the antibody or antigen-binding fragment thereof that specifically binds to AXL may be administered in combination with chemotherapy (temozolomide, platinum agents, etoposide and/or lomustine) and/or targeted therapy (bevacizumab and/or dabrafenib+trametinib) and/or pembrolizumab.

In some embodiments of the method of treatment or of use, the antibody or antigen-binding fragment thereof that specifically binds to AXL may be administered in combination with tyrosine kinase inhibitors (imatinib and/or dasatinib).

In some embodiments of the method of treatment or of use, the antibody or antigen-binding fragment thereof that specifically binds to AXL may be administered in combination with chemotherapy (vincristine, and/or doxorubicin derivatives).

In some embodiments of the method of treatment or of use, the antibody or antigen-binding fragment thereof that specifically binds to AXL may be administered in combination with chemotherapy (cytarabine and/or doxorubicin derivatives).

In some embodiments of the method of treatment or of use, the antibody or antigen-binding fragment thereof that specifically binds to AXL may be administered in combination with chemoimmunotherapy (fludarabine, cyclophosphamide and/or rituximab) and/or targeted therapy (venetoclax).

In some embodiments of the method of treatment or of use, the antibody or antigen-binding fragment thereof that specifically binds to AXL may be administered in combination with an immune checkpoint inhibitor (pembrolizumab) and/or targeted therapy (PARP inhibitors).

In some embodiments of the method of treatment or of use, the antibody or antigen-binding fragment thereof that specifically binds to AXL may be administered in combination with chemotherapy (irinotecan and/or docetaxel) and/or an immune checkpoint inhibitor (nivolumab or pembrolizumab) and/or trastuzumab.

In some embodiments of the method of treatment or of use, the antibody or antigen-binding fragment thereof that specifically binds to AXL may be administered in combination with an immune checkpoint inhibitor (nivolumab and/or pembrolizumab).

In some embodiments of the method of treatment or of use, the antibody or antigen-binding fragment thereof that specifically binds to AXL may be administered in combination with chemotherapy (cisplatin and/or gemcitabine) and/or an immune cl checkpoint inhibitor (atezolizumab and/or pembrolizumab).

In some embodiments of the method of treatment or of use, the antibody or antigen-binding fragment thereof that specifically binds to AXL may be administered in combination with chemotherapy (fluoropyrimidine and/or irinotecan) and/or targeted therapy (bevacizumab, cetuximab and/or dabrafenib+trametinib) and/or an immune checkpoint inhibitor (pembrolizumab, nivolumab and/or ipilimumab).

In some embodiments of the method of treatment or of use, the antibody or antigen-binding fragment thereof that specifically binds to AXL may be administered in combination with chemotherapy (cisplatin and/or gemcitabine) and/or nivolumab.

In some embodiments of the method of treatment or of use, the antibody or antigen-binding fragment thereof that specifically binds to AXL may be administered in combination with chemotherapy (methotrexate and/or ifosfamide).

In some embodiments of the method of treatment or of use, the antibody or antigen-binding fragment thereof that specifically binds to AXL may be administered in combination with chemotherapy (vincristine and/or docorubicin).

In some embodiments of the method of treatment or of use, the antibody or antigen-binding fragment thereof that specifically binds to AXL may be administered in combination with chemotherapy (paclitaxel, cisplatin and/or gemcitabine) and/or pembrolizumab.

In some embodiments of the method of treatment or of use, the antibody or antigen-binding fragment thereof that specifically binds to AXL may be administered in combination with an immune checkpoint inhibitor (nivolumab and/or pembrolizumab) and/or trastuzumab and/or targeted therapy (BRAF/MEK inhibitors).

In some embodiments of the method of treatment or of use, the antibody or antigen-binding fragment thereof that specifically binds to AXL may be administered in combination with central acetylcholinesterase inhibitors (donepezil, rivastigmine and/or galantamine).

In some embodiments of the method of treatment or of use, the antibody or antigen-binding fragment thereof that specifically binds to AXL may be administered in combination with nintedanib.

In some embodiments of the method for treating or the use, a suitable dose of the monoclonal antibody or antigen-binding fragment thereof that specifically binds to AXL according to the present invention will range from 0.1 to 200 mg/kg.

### BRIEF DESCRIPTION OF DRAWINGS

FIG. 1 is a genetic map of the vector pIntA_CK_01_004 bearing the sequence of the light chain of anti-AXL antibody 01_004.

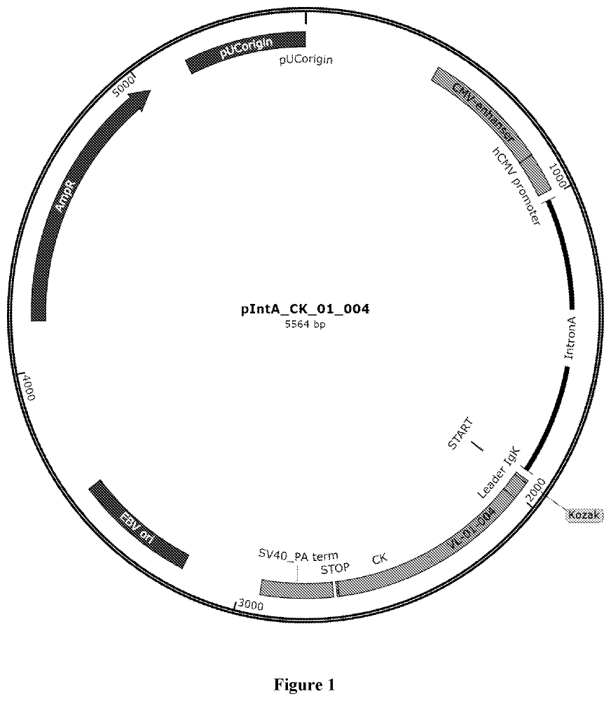

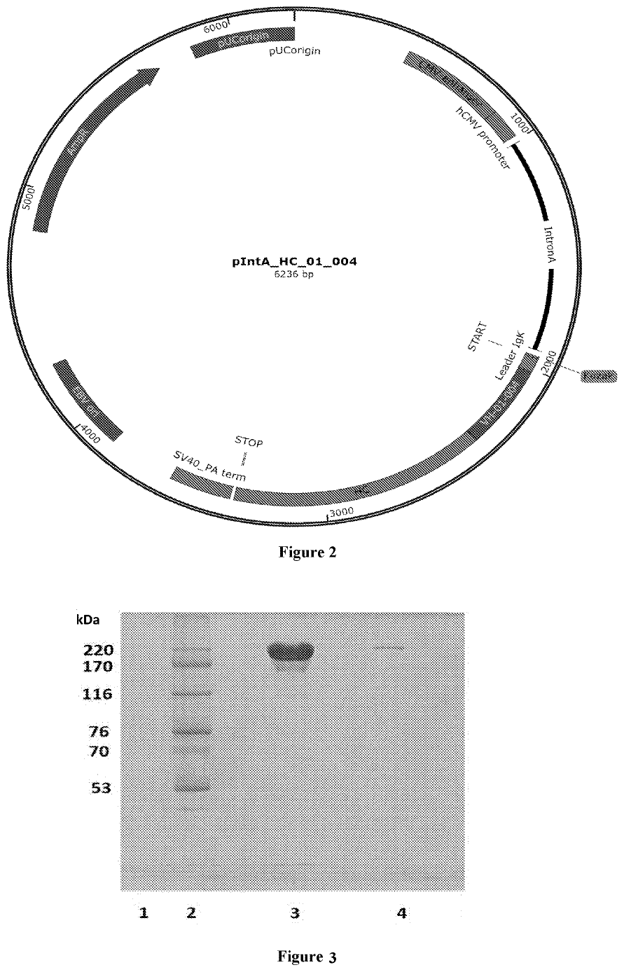

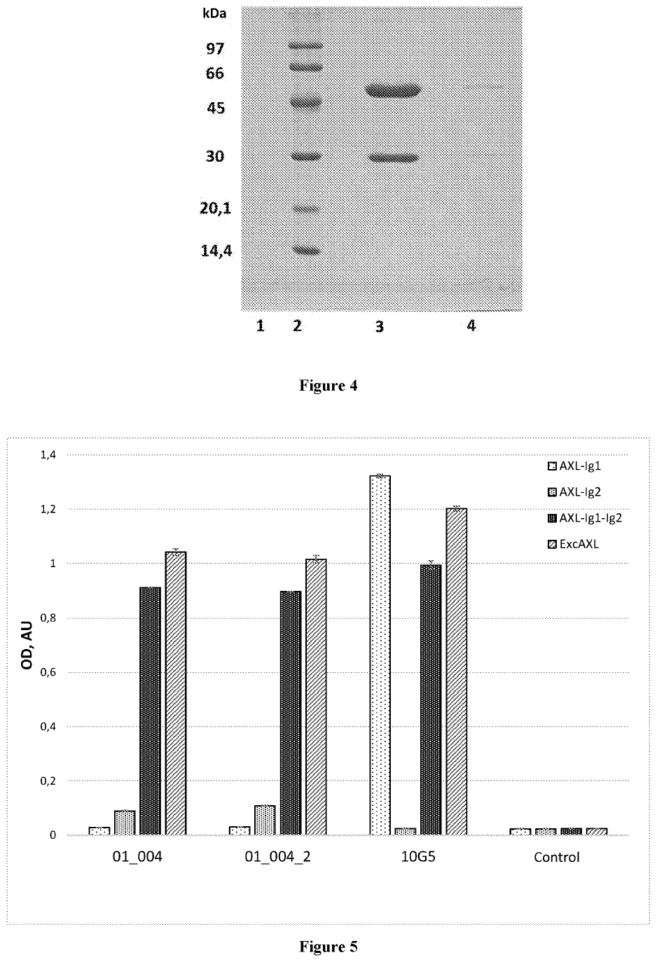

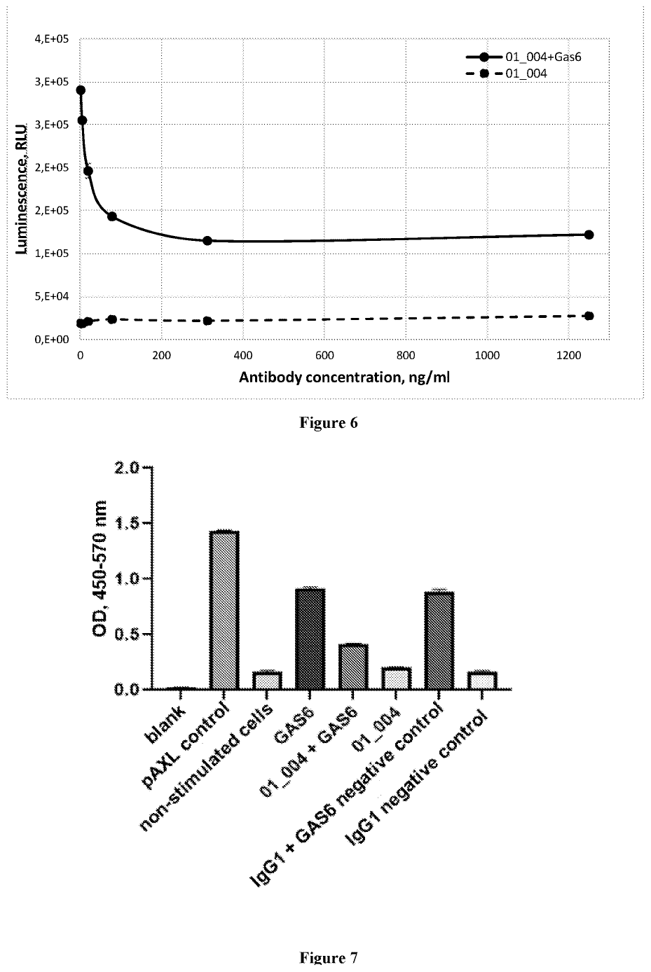

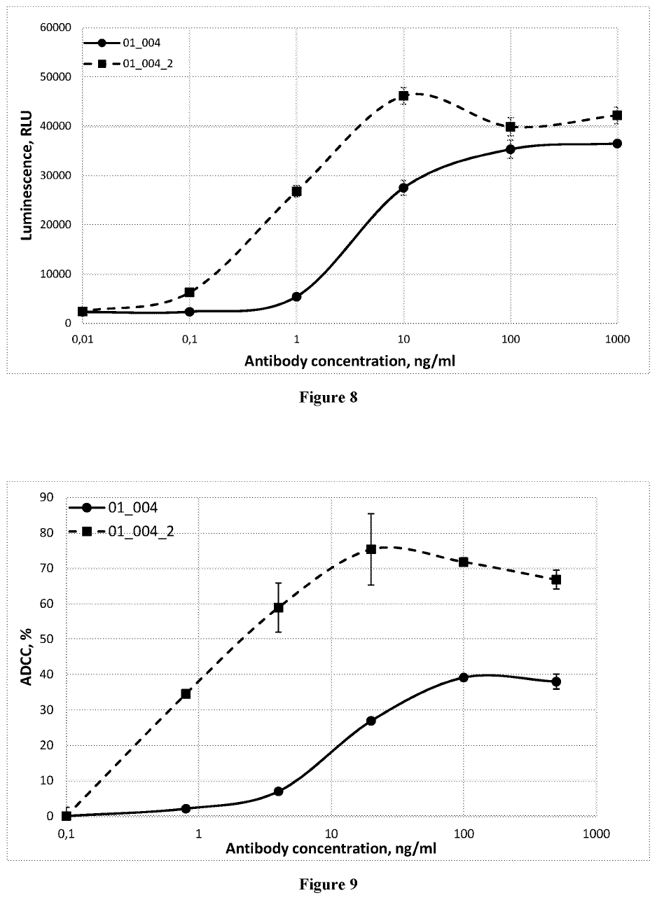

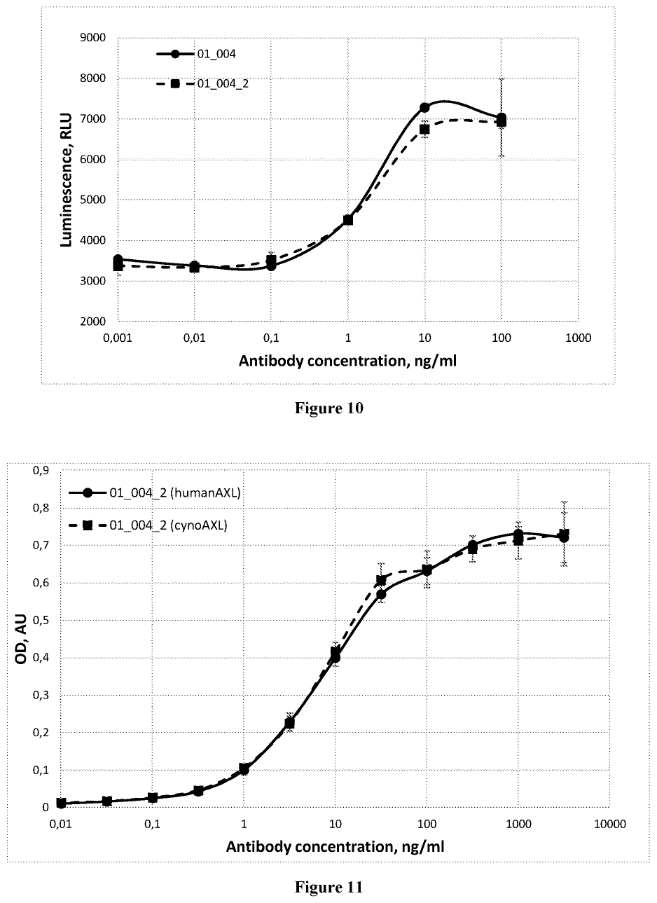

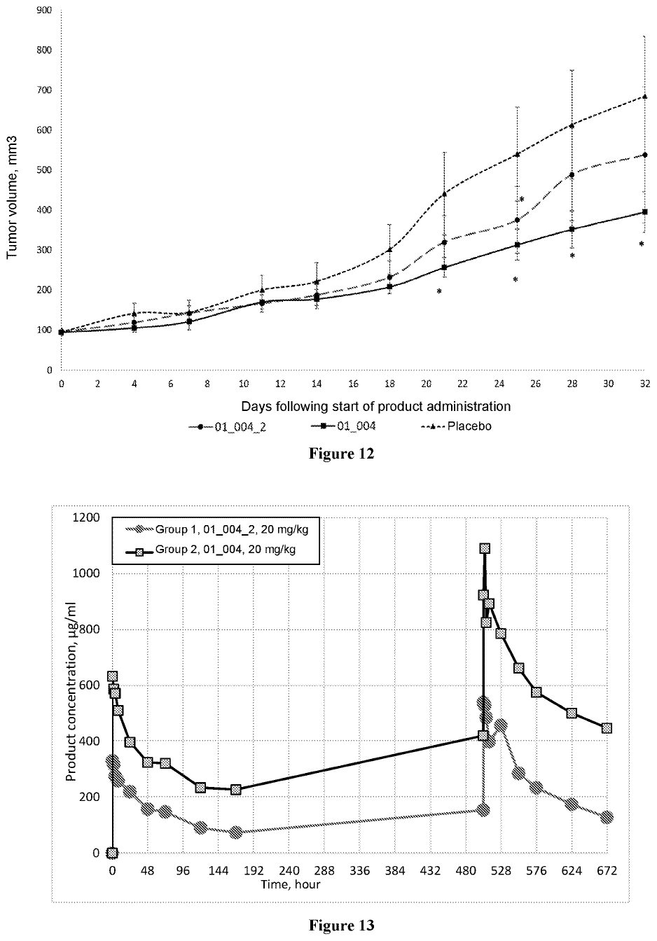

FIG. 2 is a genetic map of the vector pIntA_HC_01_004 bearing the sequence of the heavy chain of anti-AXL antibody 01_004.
For FIGS. 1 to 2

| Name | Definition |
|---|---|
| CMV-promotor | Eukaryotic promoter of human |
| | cytomegalovirus |
| CMV enhancer | Enhancer sequence of human |
| | cytomegalovirus |
| Intron A | Acceptor site |
| Kozak | Kozak consensus sequence for translation |
| | initiation |
| START | Start codon |
| Leader IgK | Murine IgK signal leader peptide |
| VL-01_004 | Gene of the variable domain of the light |
| | chain of anti-AXL antibody 01_004 |
| CK | Sequence of k light chain constant domain of |
| | IgGI antibody |
| VH-01_004 | Gene of the variable domain of the heavy |
| | chain of anti-AXL antibody 01_004 |
| HC | Sequence of HC heavy chain constant |
| | domains of IgG1 antibody |
| STOP | Stop codon |
| SV40 PA term | Transcription terminator, polyadenylation |
| | signal of SV40 virus |
| EBV ori | Origin site of Epstein-Barr virus replication |
| AmpR | Beta-lactamase gene that confers resistance |
| | to ampicillin. It enables selection of E.coli |
| | cell culture |
| pUCorigin | High-copy bacterial origin of replication |

FIG. 3 is an electrophoregram of candidate 01_004 obtained using vertical gel electrophoresis under denaturing non-reducing conditions in 8% PAAG.
1—buffer solution for applying samples, free of 2-mercaptethanol;
2—molecular protein markers; molecular weights corresponding to the lanes (kDa) are shown to the left of the electrophoregrams;
3—01_004, 10 μg load;
4—01_004, 0.1 μg load.

FIG. 4 is an electrophoregram of candidate 01_004 obtained using vertical gel electrophoresis under denaturing reducing conditions in 12.5% PAAG.
1—buffer solution for applying samples, comprising 2-mercaptethanol;
2—molecular protein markers; molecular weights corresponding to the lanes (kDa) are shown to the left of the electrophoregrams;
3—01_004, 10 μg load;
4—01_004, 0.1 μg load.

FIG. 5 is a graph illustrating ELISA results to measure binding of anti-AXL antibodies to distinct extracellular AXL fragments.
10G5—control anti-AXL antibody to AXL-Ig1 domain
Control—control without introducing anti-AXL antibodies

FIG. 6 is a graph illustrating the inhibiting activity of anti-AXL antibodies in an assay using a reporter cell line.

FIG. 7 is a graph illustrating ELISA results to measure inhibition of GAS6-dependent phosphorylation of AXL by anti-AXL antibody.
blank-control without introducing cell lysate, background level of OD in ELISA
pAXL Control—phosphorylated AXL control product from the DuoSet IC ELISA intracellular Human Phospho-Axl kit (R&D Systems)
non-stimulated cells—cells incubated in the absence of GAS6 and antibodies
GAS6—cells incubated in the presence of GAS6
IgG1 negative control—cells incubated in the presence of a control (not anti-AXL) IgG1 antibody
01_004—cells incubated in the presence of antibody 01_004
01_004+GAS6—cells incubated in the presence of GAS6 and antibody 01_004
IgG1+GAS6 negative control—cells incubated in the presence of GAS6 and a control (not anti-AXL) IgG1 antibody

FIG. 8 is a graph illustrating antibody-dependent cellular cytotoxicity (ADCC) in an assay using a reporter cell line.

FIG. 9 is a graph illustrating antibody-dependent cellular cytotoxicity (ADCC) in an assay using PBMCs.

FIG. 10 is a graph illustrating antibody-dependent cellular phagocytosis (ADCP) in an assay using a reporter cell line.

FIG. 11 is a graph illustrating dose-dependent binding of anti-AXL antibodies to human and Cynomolgus AXL.

FIG. 12 is a graph illustrating tumor volumes vs time following the start of administration of anti-AXL drugs to mice during in vivo study on a subcutaneous xenograft model.
Note: *p<0.05, statistically significant difference from the control (Mann-Whitney test).

FIG. 13 is a graph illustrating change in concentration of candidate anti-AXL products in blood serum of monkeys following repeated intravenous administration.

### EXAMPLES

The following examples are provided for better understanding of the invention. These examples are for purposes of illustration only and are not to be construed as limiting the scope of the invention in any manner.

### Materials and General Methods

General information regarding the nucleotide sequences of human immunoglobulin light and heavy chains is given in: Kabat, E. A., et al., Sequences of Proteins of Immunological Interest, 5th ed., Public Health Service, National Institutes of Health, Bethesda, MD (1991). Amino acids of antibody chains are numbered according to the EU numbering scheme (Edelman, G. M., et al., Proc. Natl. Acad. Sci. USA 63 (1969) 78-85; Kabat, E. A., et al., Sequences of Proteins of Immunological Interest, 5th ed., Public Health Service, National Institutes of Health, Bethesda, MD, (1991).

### Recombinant DNA Techniques

Standard methods were used to manipulate DNA as described in Sambrook, J. et al, Molecular cloning: A laboratory manual; Cold Spring Harbor Laboratory Press, Cold Spring Harbor, New York, 1989. The molecular biological reagents were used according to the manufacturer protocols.

### Gene Synthesis

Desired gene segments were prepared from oligonucleotides made by chemical synthesis. The gene segments of 300-1400 bp long, which were flanked by singular restriction sites, were assembled by annealing and ligation of oligonucleotides including PCR amplification and subsequently cloned via the restriction sites. The DNA sequences of the subcloned gene fragments were confirmed by DNA sequencing.

### DNA Sequence Determination

DNA sequences were determined by Sanger sequencing.

### DNA and Protein Sequence Analysis and Sequence Data Management

The Unipro's UGENE suite version 1.29 and SnapGene version 6.1 were used for sequence creation, mapping, analysis, annotation and illustration.

### Expression Vectors

For the expression of the antibodies described in the application materials, variants of expression plasmids intended for expression of antibodies in prokaryotic cells (E. coli), transient expression in eukaryotic cells (e.g., in CHO cells) were applied. Beside the antibody expression cassette the vectors contained: an origin of replication which allows replication of said plasmid in E. coli, genes which confer resistance in E. coli to various antibiotics (e.g. to ampicillin, kanamycin).

The fusion genes comprising the described antibody chains as described below were generated by PCR and/or gene synthesis and assembled with known recombinant methods and techniques by connection of the according nucleic acid segments, e.g. using unique restriction sites in the corresponding vectors. The subcloned nucleic acid sequences were verified by DNA sequencing. For transient transfections, larger quantities of the plasmids were prepared by plasmid preparation from transformed E. coli cultures.

### Example 1. Design and Creation of Genetic Constructs for Synthesis of Full-Length Anti-AXL Monospecific Antibodies

Monospecific anti-AXL antibodies were produced in a full-length bivalent IgG1 format. In the sequence of the Fc portion of the heavy chain of anti-AXL antibodies, two C-terminal amino acids GK were removed. Removal of two C-terminal amino acids GK in the heavy chain Fc portion sequence results in increased productivity and homogeneity of the antibody product generated in CHO cells, improved acid-base profile (ABP) thereof, wherein such deletion does not affect the pharmacokinetics, activity and stability of the antibody.

Antibodies 01-004 and 01-004_2 have the same variable domains of the light and heavy chain. Antibody 01-004_2 comprises, in the Fc portion of the heavy chain, additional amino acid substitutions S239D and I332E (according to EU numbering).

Genetic constructs were created as follows. Generated were PCR products comprising sequences of variable domains of heavy and light chains of antibodies using PCR primers comprising additional sequences complementary to the respective sites of the respective pIntA expression vectors. The below assembly procedure was performed by ligation-independent method using 3′-5′ exonuclease activity of Pfu Ultra II DNA polymerase. The nucleotide sequence of the variable domain of the light chain (VL) was inserted into the vector pIntA_CK using SapI restriction sites. The vector pIntA_CK_01_004 (FIG. 1) comprises a nucleotide sequence encoding the constant portion of the light kappa chain of IgG1 antibody. The nucleotide sequence of the variable domain of the heavy chain (VH) was inserted into the vector pIntA_HC using SapI restriction sites. The vector pIntA_HC_01_004 (FIG. 2) comprises a nucleotide sequence encoding the IgG1 antibody heavy chain constant portion comprising a deletion of the C-terminal amino acids GK. In the case of antibody 01-004_2, into the nucleotide sequence of the constant portion of the heavy chain there have been further introduced nucleotide substitutions providing for amino acid substitutions S239D and I332E.

The nucleotide sequences of the resulting plasmid constructs were confirmed using DNA sequencing. The resulting plasmids were generated in desired quantities in E. coli cells and purified using a plasmid DNA isolation kit. The plasmids were then used to transiently produce proteins in the CHO-K1-S cell line.

### Example 2. Generation, Isolation and Purification of Monoclonal Antibodies

To generate antibodies in the transient expression system used were CHO-K1-S line cells. For the generation, used was a CHO cell culture medium further supplemented with 4 mM glutamine, 10 μg/ml gentamicin, 10 μg/ml ciprofloxacin and 100 μM deoxy-2-fluoro-L-fucose. Cells were cultured in baffled flasks in orbital shaker-incubators at +37° C. in the presence of 5% CO₂ while constantly stirring.

For transient expression, a cell culture at 1.8-2·10⁶ cells/ml were transfected using linear polyethylenimine. DNA/PEI ratio was 1:7. On day 7 of culturing, the cell suspension was clarified by centrifugation and then filtered through a 0.22 μm filter.

On day 7 following transfection, samples of cellular fluid were taken and the concentration of the generated protein was measured therein by biolayer interferometry on the OctetRed 96 (ForteBio) instrument on Protein-A Biosense chips according to the manufacturer's instructions (Table 1).

TABLE 1
Productivity of antibodies to AXL

| Antibody | Protein productivity, mg/l |
|---|---|
| 01_004 | 304 |
| 01_004_2 | 157 |

While being transiently generated, antibodies 01_004 and 01_004_2 demonstrated productivity typical for monoclonal antibodies.

Antibody purification was performed by affinity chromatography using a sepharose-based sorbent with immobilized protein A. If and where necessary, additional chromatographic purification of the product was performed on the Ceramic Hydroxyapatite Type I sorbent. Quality of the resulting antibodies was analysed using electrophoresis in denaturing (in the presence of sodium dodecyl sulfate) polyacrylamide gel under non-reducing conditions (without the addition of mercaptoethanol) and under reducing conditions (with the addition of mercaptoethanol) (FIGS. 3-4).

### Example 3. Production of Genetic Constructs to Synthesize Antigens Generation, Isolation and Purification of Antigens

To obtain various fragments of the AXL receptor, created was a number of genetic constructs encoding various human or Cynomolgus AXL domains. Expression plasmids for generating antigen proteins in CHO cells are based on pEE or pIntA vectors and comprise nucleotide sequences encoding the respective AXL receptor fragments fused, for the convenience of subsequent purification of generated proteins, to His6 tag or Ig1 antibody Fc portion-based tag. Genetic constructs were produced using conventional genetic engineering techniques.

Data on the human AXL amino acid sequence were obtained from the NCBI database (https://www.ncbi.nlm.nih.gov/protein/AAA61243.1?report-genbank&log$=protalign&blast_rank=1&RI D-SV8VAB28013). The human AXL extracellular portion sequence (417AA) is represented by SEQ ID NO: 23.

The following antigens were obtained:
ExcAXL—a human AXL receptor fragment, comprising all AXL extracellular domains, fused to His-tag (SEQ ID NO: 24).
AXL-Ig1—a human AXL receptor fragment, comprising the sequence of the extracellular domain Ig-like C2-type 1 of AXL, fused using a linker to IgG1 Fc (SEQ ID NO: 25).
AXL-Ig2—a human AXL receptor fragment, comprising the sequence of the extracellular domain Ig-like C2-type 2 of AXL, fused using a linker to IgG1 Fc (SEQ ID NO: 26).
AXL-Ig1-Ig2—a human AXL receptor fragment, comprising the sequences of the extracellular domains Ig-like C2-type 1 and 2 of AXL, fused using a linker to IgG1 Fc (SEQ ID NO: 27).

The gene encoding the Cynomolgus macaque AXL (Macaca fascicularis) was amplified from a product of cDNA derived from RNA from the Cynomolgus macaque smooth muscle. Next, the gene sequence was determined by sequencing. The Cynomolgus AXL extracellular portion sequence obtained in this fashion was used to create a respective expression plasmid construct.
cynoAXL—a Cynomolgus AXL receptor fragment, comprising sequences of all extracellular domains of the macaque AXL receptor, fused using a linker to His6- and FLAG-tags (SEQ ID NO: 28).

The nucleotide sequences of the resulting plasmid constructs were confirmed using DNA sequencing. The resulting plasmids were generated in desired quantities in E. coli cells and purified using a plasmid DNA isolation kit. The plasmids were then used to transiently produce proteins in the CHO-K1-S cell line.

To generate antigens in the transient expression system used were CHO-K1 line cells. Cells were cultured in baffled flasks in orbital shaker-incubators at +37° C. in the presence of 5% CO₂ while constantly stirring.

For transient expression, a cell culture at 1.8-2·10⁶ cells/ml were transfected using linear polyethylenimine. DNA/PEI ratio was 1:7. On day 7 of culturing, the cell suspension was clarified by centrifugation and then filtered through a 0.22 μm filter.

Antigens comprising Fc-tag were purified similarly to antibodies on a protein A affinity sorbent (See example 2).

Antigens comprising Fc-tag were purified using affinity chromatography on a sorbent with immobilized protein A. Antigens comprising HIS6-tag were purified using metal chelate chromatography on a Ni-charged sorbent. The quality of the resulting antigens was analysed using electrophoresis in a denaturing 4-15% gradient polyacrylamide gel under non-reducing or reducing conditions. The analysis of the proper quality of the antigens obtained was confirmed by electrophoresis in a denaturing 4-15% gradient polyacrylamide gel under non-reducing conditions without the addition of mercaptoethanol and under reducing conditions with the addition of mercaptoethanol.

### Example 4. Test to Evaluate Binding of Anti-AXL Antibodies to Human AXL Domains Using ELISA

Antigens (ExcAXL, AXL-Ig1, AXL-Ig2, AXL-Ig1-Ig2) were immobilized in wells of a high-absorption 96-well plate. To this end, to the wells of the plate was introduced 100 μl of antigen solution at a concentration of 1 μg/ml in PBS, the plate was incubated for 16 hours at room temperature.

Next, the liquid was removed from the wells, the wells were washed with 300 μl of washing buffer solution (1×PBS pH 7.3-7.5, 0.05% Tween-20) and 300 μl/well of blocking buffer solution was added (1×PBS with 1% BSA). The plate was incubated for 1 hour at room temperature. Liquid was removed again and the plate was washed. Then, to the wells of the plate were introduced 100 μl of test antibodies at a concentration of 1 μg/ml in a dilution buffer (1×PBS with 0.1% BSA, 0.05% Tween-20). Next, the plate was incubated with antibodies on a thermal shaker for 1 hour at 37° C., 600 rpm. After incubation, liquid was removed, the plate was washed and 100 μl of anti-human Fab-HRP antibodies in a dilution buffer were added. After incubation on a thermal shaker at 37° C. and 600 rpm, the plate was washed for an hour and 100 μl of reaction substrate (0.083 mg/ml TMB, 0.02% H₂O₂) in an acetate buffer solution (0.05M, pH 5.5) was added. The plate was incubated at room temperature for 10-15 minutes. The reaction was stopped by adding 50 μl of 10% H₂SO₄ to each well of the plate. Optical density values were measured at a wavelength of 450 using 540 nm wavelength correction by means of a plate reader.

ELISA results (FIG. 5) show that the reproduced anti-AXL antibody 10G5 binds to AXL fragments, comprising a distinct Ig-like C2-type 1 domain of AXL (AXL-Ig1) or two Ig-like C2-type 1 and Ig-like C2-type 1 domains (AXL-Ig1-Ig2), and to complete extracellular portion of AXL (ExcAXL) but do not bind to AXL fragments that comprise a distinct Ig-like C2-type 2 domain (AXL-Ig2). Anti-AXL antibodies 01_004 and 01_004_2 bind to AXL fragments, comprising a distinct Ig-like C2-type 2 domain of AXL (AXL-Ig2) or two Ig-like C2-type 1 and Ig-like C2-type 1 domains (AXL-Ig1-Ig2), and to complete extracellular portion of AXL (ExcAXL) but do not bind to AXL fragments that comprise a distinct Ig-like C2-type 1 domain (AXL-Ig1).

### Example 5. Test to Determine Blocking Activity of Anti-AXL Antibodies Using Reporter Cell Line

The test used a reporter cell line derived from HEK293 cells, stably surface-expressing AXL and comprising a gene encoding firefly luciferase under the control of the STAT3-STAT5-API promoter.

The assay was performed in a 96-well culture plate designed for luminescence assays. The suspension contained per well 30,000 cells of the reporter cell line, test antibody at a concentration as indicated in the graph in the presence or absence of Gas6. The final volume of the cell suspension in a well was 100 μl, all components of the suspension were prepared in DMEM medium free of fetal bovine serum. After adding all the components, the plate was incubated for 16 hours at 37° C., 5% CO₂; then, using a luminescence assay kit, the luminescence intensity in the wells was measured. The measurement was carried out using a plate reader.

Test results (FIG. 6) show that the anti-AXL antibody 01_004 induces suppression of GAS6-dependent activation of intracellular AXL signaling.

### Example 6. Test to Evaluate Inhibition of GAS6-Dependent Phosphorylation of AXL

AXL phosphorylation was evaluated using ELISA. Suspension of H1299 cells in a volume of 3 ml was introduced into a 6-well flat-bottomed plate at a seeding dose of 1×10⁴ cells/cm² in a complete growth medium (DMEM/F12, 2 mM L-glutamine, 10% FBS HI), the plate was incubated for 24 hours at 37° C., 5% CO₂. After incubation, the growth medium was removed from the wells and a serum-free medium (DMEM/F12, 2 mM L-glutamine) was introduced at 2 ml/well, the wells were incubated overnight at 37° C., 5% CO₂. After incubation, the serum-free medium was replaced with a fresh one at 2 ml/well, 500 ml/well of 01_004 or control IgG1 antibody was added at a concentration of 500 μg/ml in two replicates (an equivalent volume of serum-free medium was added to the wells without antibodies), the wells were incubated for an hour at 37° C., 5% CO₂. After incubation, 500 μl of GAS6 was added to one of replicates of each sample at a concentration of 3 μg/ml, 500 μl of serum-free medium was added to the other one, the samples were incubated for 30 minutes at 37° C., 5% CO₂. After incubation, the medium was removed from the wells of the plate and the cell monolayer was washed twice with ice-cold PBS at 2 ml/well, 300 μl/well of a lysing buffer with phosphatase and protease inhibitors was introduced, the plate was incubated on ice for 45 minutes. Concentration of total protein in samples was determined by BCA assay according to the manufacturer's instructions. Cell lysates were stored at −80° C.

Next, ELISA was performed using a commercial kit, DuoSet IC ELISA intracellular Human Phospho-Axl, (R&D Systems) according to the manufacturer's instructions. Before being introduced to wells of the experimental plate, cell lysates were diluted to a concentration of 300 μg/ml.

Test results (FIG. 7) show that anti-AXL antibody 01_004 induces suppression of GAS6-dependent phosphorylation of AXL.

### Example 7. Antibody-Dependent Cellular Cytotoxicity (ADCC) Assay Using Reporter Cell Line

The assay used a reporter cell line Jurkat-NFAT-Luc-CD16 created on the basis of the Jurkat cell line, stably expressing CD16 on the surface and containing a gene encoding firefly luciferase, under the control of the NFAT promoter; the AXL-expressing line, NCI-H1299, was used as target cells. Jurkat-NFAT-Luc-CD16 and NCI-H1299 cells were cultured at 37° C. with 5% CO₂ in RPMI-1640 nutrient medium supplemented with 10% fetal bovine serum.

The assay was performed in a 96-well culture plate designed for luminescence assays. Each well with suspension contained 25,000 Jurkat-NFAT-Luc-CD16 effector cells, 25,000 target NCI-H1299 cells expressing AXL, as well as test antibodies at a concentration as indicated in the graph. The final volume of the cell suspension and antibodies in a well was 100 μl, all components of the suspension were prepared in RPMI-1640 medium comprising 10% FBS. After adding all the components, the plates were incubated for 16 hours at 37° C., 5% CO₂; then, using a luminescence assay kit, the luminescence intensity in the wells was measured. The measurement was carried out using a plate reader.

Test results (FIG. 8) show that the anti-AXL antibodies 01_004 and 01_004_2 have ADCC activity.

### Example 8. Antibody-Dependent Cellular Cytotoxicity (ADCC) Assay Using PBMCs

PBMCs were isolated from whole blood from healthy donors by Ficoll density gradient centrifugation. A549 cells were cultured at 37° C. with 5% CO₂ on a DMEM nutrient medium supplemented with 10% fetal bovine serum.

The assay was conducted in a 96-well culture plate. The suspension contained per well 30000 A549 target cells expressing AXL, 300000 freshly isolated PBMCs, as well as test antibodies at the specified concentration. The final volume of cell suspension in a well was 150 ml, all components were prepared in RPMI-1640 medium comprising 10% fetal bovine serum. The plate was incubated for 16 hours at 37° C. with 5% CO₂. At the end of incubation, to the control wells comprising only target cells was added 10× Lysis Solution (CytoTox96® Non-Radio Cytotoxicity Assay, Promega) to 1× concentration, the plate was incubated at 37° C. in a CO₂ incubator for 30 minutes. Supernatant from all wells was transferred to a V-shaped plate, centrifuged at 300 g, and 50 μl of supernatant was taken from each well into new plates. Assay Buffer and Substrate Mix (CytoTox96® Non-Radio Cytotoxicity Assay kit) were mixed and 15 μl were added to the wells containing supernatant. Plates were incubated in the dark at room temperature for up to 30 minutes. 15 ml of Stop Solution (CytoTox 96® Non-Radioactive Cytotoxicity Assay kit, Promega) was added to each well. Optical density was measured a wavelength of 490 nm using a plate reader.

Assay results (FIG. 9) show that the anti-AXL antibodies 01_004 and 01_004_2 have ADCC activity.

### Example 9. Assay to Determine Antibody-Dependent Cellular Phagocytosis (ADCP) Using Reporter Cell Line

The assay used the reporter cell line Jurkat-NFAT-Luc-CD64 created on the basis of the Jurkat cell line, stably surface-expressing CD64 and containing a gene encoding firefly luciferase, under the control of the NFAT promoter; the A549 cell line was used as target cells. Jurkat-NFAT-Luc-CD64 and A549 cells were cultured at 37° C. with 5% CO₂ on appropriate growth medium supplemented with 10% fetal bovine serum, Jurkat-NFAT-Luc-CD64 cells were cultured in RPMI-1640 medium, A549 cells were cultured in DMEM medium.

The assay was performed in a 96-well culture plate designed for luminescence assays. The suspension contained per well 30 000 Jurkat-NFAT-Luc-CD64 reporter line cells and 30000 A549 target cells, as well as test antibodies at the specified concentration. The final volume of suspension per well was 100 μl, all suspension components were prepared in RPMI-1640 medium comprising 10% fetal bovine serum. After adding all the components, the suspension was incubated for 16 hours at 37° C., 5% CO₂; then, using a commercial luminescence assay kit, the luciferase intensity in the wells was measured. The measurement was carried out using a plate reader.

The assay results (FIG. 10) show that anti-AXL antibodies 01_004 and 01_004_2 induce CD64-dependent NFAT signaling activation mediating ADCP.

### Example 10. Anti-AXL Antibody-Antigen Binding Assay Using Enzyme-Linked Immunosorbent Assay (ELISA)

Antigens (human_AXL and cynoAXL) were immobilized in wells of a high-absorption 96-well plate. To this end, to the wells of the plate was introduced 100 μl of antigen solution at a concentration of 1 μg/ml in carbonate buffer (0.1M NaHCO₃, pH 9.5), the plate was incubated for 16 hours at room temperature.

Next, the liquid was removed from the wells, the wells were washed with 300 μl of washing buffer solution (1×PBS pH 7.3-7.5, 0.05% Tween-20) and 300 μl/well of blocking buffer solution was added (1×PBS with 1% BSA). The plate was incubated for 1 hour at room temperature. Liquid was removed again and the plate was washed. Next, to wells of the plate was added 100 μl of antibody 01_004_2, at a concentration as shown in the graph, in dilution buffer (1×PBS with 0.1% BSA, 0.05% Tween-20). Next, the plate was incubated with antibodies on a thermal shaker for 1 hour at 37° C., 600 rpm. After incubation, liquid was removed, the plate was washed and 100 μl of anti-human Fab-HRP antibodies in a dilution buffer were added. After incubation on a thermal shaker at 37° C. and 600 rpm, the plate was washed for an hour and 100 μl of reaction substrate (0.083 mg/ml TMB, 0.02% H₂O₂) in an acetate buffer solution (0.05M, pH 5.5) was added. The plate was incubated at room temperature for 10-15 minutes. The reaction was stopped by adding 50 μl of 10% H₂SO₄ to each well of the plate. Optical density values were measured at a wavelength of 450 using 540 nm wavelength correction by means of a plate reader.

Assay results (FIG. 11) show that anti-AXL antibody 01_004_2 binds to human AXL and Cynomolgus AXL.

### Example 11. Determination of Aggregation Stability of Anti-AXL Antibodies Following Thermal Stress

Aggregation stability of anti-AXL antibodies was tested using thermal stress (50° C., 48 h). Proportion of monomer in samples before and following thermal stress was determined by size-exclusion HPLC.

Test samples were thermostated at 50° C. for 72 hours. After heating, centrifugation-clarified intact and stressed samples were transferred for analysis by size-exclusion HPLC (SEC HPLC) with a UV detector and by capillary isoelectric focusing method. Chromatography was performed on Agilent 1100 HPLC system on Tosoh TSK-Gel G3000SWXL column, detection was performed at a wavelength of 280 nm.

TABLE 2
Aggregation stability of anti-AXL antibodies

| | Monomer content | Monomer content |
|---|---|---|
| | before thermal | following |
| Antibody | stress, % ± SD | thermal stress, % ± SD |
| 01_004 | 98.17 ± 0.06 | 97.9 ± 0.1 |
| 01_004_2 | 97.3 ± 0.7 | 94.6 ± 0.4 |

Conclusion: antibodies to AXL according to the invention have high aggregation stability.

### Example 12. Determination of Physico-Chemical Stability of Anti-AXL Antibodies in Human Blood Serum

Comparative study of stability of anti-AXL antibodies 01_004 and 01_004_2 in human and mouse blood serum was conducted. Pooled blood serum was obtained from 7 healthy donors and 7 healthy outbred mice, mixed in equal proportions by volume. Test antibodies were added to serum with 0.1% sodium merthiolate to a concentration of 25 μg/ml and incubated for 14 days at a temperature of 37° C. Next, the concentration of anti-AXL antibodies was determined using enzyme immunoassay in human and mouse serum before and following incubation for 14 days at 37° C., and stability % was calculated. The results are shown in Table 3.

TABLE 3
Stability data for anti-AXL antibodies.

| | Stability, % | |
|---|---|---|
| Antibody | human serum | mouse serum |
| 01_004_2 | 100 | 72 |
| 01_004 | 89 | 61 |

Detectable concentration of anti-AXL antibody following storage in human serum +37° C. for 14 days at a concentration of 25 μg/ml corresponds to high stability. Normal stability of monoclonal antibodies in human serum is considered to be 60%.

### Example 13. Determination of Constants of Binding of Antibody 01_004 to Various Antigens

Constants of binding of antibody 01_004 to various antigens were determined by SPR (surface plasmon resonance) using Biacore 8K (Cytiva). The test antigen was immobilized on Sensor Chip CM5 (Cytiva), followed by analysis of binding thereof to anti-AXL antibody 01_004 in PBS 0.02% Tween-20 pH 7.4. Measurement data was processed using the Biacore Insight Evaluation (Cytiva) software.

Mean values of equilibrium binding constants are shown in Table 4.

TABLE 4
Equilibrium constants of binding of antibody
01_004 to various antigens

| | Results | KD (M) |
|---|---|---|
| 1 | Human AXL | 4.30E−11 |
| 2 | cynomolgus (Macaca fascicularis) AXL | 5.28E−11 |
| 3 | rhesus (Macaca mulatta) AXL | 1.05E−10 |
| 4 | mouse (Mus musculus) AXL | no binding detected |
| 5 | rat (Rattus norvegicus) AXL | no binding detected |
| 6 | rabbit (Oryctolagus cuniculus) AXL | 2.40E−7 |
| 7 | TYRO3 | no binding detected |
| 8 | MERTK | no binding detected |

Conclusion: antibody to AXL according to the invention specifically binds to the human AXL antigen, as well as to the monkey AXL antigen, but does not bind to other tested closely related antigens.

### Example 14. Determination of Anti-Tumor Activity of Anti-AXL Products Using a Subcutaneous Xenograft Model

Antitumor activity of the products was measured using a subcutaneous tumor xenograft model. To this end, female BALB/c Nude mice were subcutaneously transplanted with 5×10⁶ tumor A549 line cells mixed with Matrigel® in 1:1 ratio. After reaching a mean tumor volume of 95.6±1 mm³, the mice were divided into 3 groups of 9 mice each so that the mean tumor volumes between the groups were comparable.

01-004, 01-004_2 products were administered intraperitoneally at a dose of 20 mg/kg twice a week for 4 weeks. The negative control group was injected with a buffer solution of anti-AXL products in a similar mode.

During the experiment, the linear dimensions of the tumor were determined. Tumor volume was calculate using the formula V=L×W×H×π/6, where L and W are the large and small diameters of the tumor, respectively, H is the height of the tumor.

The efficacy of test product was measured by the index of tumor growth inhibition (TGI) calculated according to the following formula:

$$TGI ( \% ) = ( {V}_{c} - {V}_{p} ) / {V}_{c} \times 100 ,$$

where V_{c} and Vₚ are median tumor volumes in the negative control group and the group of animals receiving the test product, respectively. The higher the index value, the more pronounced the antitumor effect.

On day 32 of the experiment, the animals were euthanized, tumors were extracted and weighed using analytical scales.

FIG. 12 shows the dynamics of tumor volume.

At the time of the start of product administration, the tumor volume did not differ significantly between the groups. In the group of animals receiving the 01_004_2 product, on day 25 of the experiment, the tumor volume was significantly less than that in the control group. In the case of repeated administration of the 01_004 product, the tumor volume was significantly less than that in the control group from day 21 to day 32 of the experiment.

The TGI index on day 32 of the experiment was as follows: 35% for the 01_004_2 product group, 43% for the 01_004 product group.

Table 5 shows median values of tumor masses at the end of the experiment.

TABLE 5
Tumor masses on day 32 of experiment

| Product | Mass, median (mg) |
|---|---|
| 01_004_2 | 349 |
| 01_004 | 326 |
| Placebo | 624 |

There was a tendency for this parameter to decrease in the experimental groups when compared to the control.

### Example 15. Determination of Toxicity and Local Irritant Effect of Anti-AXL Products in Case of Repeated Intravenous Administration to Cynomolgus Monkeys (Macaca fascicularis) for 4 Weeks

Toxicity and local irritant effect were measured on Cynomolgus monkeys. 01_004_2 and 01_004 products were administered intravenously at a dose of 20 mg/kg once a week for 4 weeks.

In the course of the experiment, we monitored body weight, motor activity, vegetative reactions, coat condition, eye changes (palpebral fissure/conjunctiva/sclera conditions), condition of mucous membranes of oral and nasal cavities, stool parameters, appetite, respiration parameters, functional state of the excretory system; we analyzed the cellular composition of peripheral blood, evaluated the state of the coagulation system, protein, carbohydrate and enzymatic liver function (biochemical parameters of blood serum: activity of indicator enzymes, total bilirubin, cholesterol, total protein, triglycerides, glucose), condition of the urinary system according to the results of biochemical analyses of animal serum (according to the level of urea, creatinine, sodium and potassium ions and the results of urine analysis), we conducted a pathomorphological examination of internal organs. The local irritant effect was evaluated according to visual examination and histological study of the site of administration of products and draining lymph nodes.

The results of the experiment showed that the administration of 01_004_2 and 01_004 products was well tolerated by the animals. During the entire observation period, we did not observe any changes in vegetative reactions, coat condition, eyes (conjunctiva, sclera, lacrimation, secretion), mucous membranes of nasal and oral cavities; stool, respiration, decreased appetite parameters, and in animal behavior.

01_004_2 and 01_004 products had no effect on body weight, coagulation system, hematological parameters, protein, carbohydrate and enzymatic liver functions, as well as the urinary system of Cynomolgus monkeys.

The results of pathomorphological examinations show no abnormalities in the structure of organs and tissues of experimental animals associated with the administration of test products.

Following repeated intravenous administration for 4 weeks at a dose of 20 mg/kg, 01_004_2 and 01_004 products did not have a local irritant effect.

The resulting data show that, following repeated intravenous administration for 4 weeks, 01_004_2 and 01_004 products at a dose of 20 mg/kg had no effect on the main organs and organ systems of Cynomolgus monkeys (Macaca fascicularis).

### Example 16. Determination of Pharmacokinetics of Anti-AXL Products Following Repeated Intravenous Administration to Cynomolgus Monkeys (Macaca fascicularis)

This study was conducted as part of analysis of toxicity of anti-AXL products in primates (see example 15).

To assess the pharmacokinetic parameters, blood from all animals was collected immediately before the first administration of the 01_004_2 product or 01_004 product (background), and then at time points of 0, 25, 2, 4, 8, 24, 48, 72, 120, 168 h following the first administration of the 01_004_2 product or 01_004 product, as well as immediately before the 4th administration of the product (504 hours) and at time points of 504, 25, 506, 508, 512, 528, 552, 576, 624, 672 h following the 4th administration of the product.

Concentration of antibodies in blood serum was measured by solid-phase enzyme immunoassay (ELISA) using horseradish peroxidase as an indicator enzyme. To determine the concentration of anti-AXL antibodies in the samples, used were solutions of the corresponding anti-AXL antibodies with a known concentration to construct a calibration curve.

FIG. 13 shows mean curves for changes in concentrations of antibodies 01_004_2 and 01_004 over time in the blood serum of monkeys. The results showed that following repeated intravenous administration of anti-AXL antibody products at a dose of 20 mg/kg, the mean values of PK parameters are typical of therapeutic monoclonal antibodies. Also, the resulting data show the presence of accumulation of test antibodies.

1. A monoclonal antibody or antigen-binding fragment thereof that specifically binds to AXL, comprising:
(a) a light chain variable domain comprising:
CDR1 with the amino acid sequence of SEQ ID NO: 1,
CDR2 with the amino acid sequence of SEQ ID NO: 2 and
CDR3 with the amino acid sequence of SEQ ID NO: 3; and
(b) a heavy chain variable domain comprising:
CDR1 with the amino acid sequence of SEQ ID NO: 7,
CDR2 with the amino acid sequence of SEQ ID NO: 8 and
CDR3 with the amino acid sequence of SEQ ID NO: 9.

2. The monoclonal antibody or antigen-binding fragment thereof according to claim 1, wherein the light chain variable domain comprises the amino acid sequence of SEQ ID NO: 13.

3. The monoclonal antibody or antigen-binding fragment thereof according to claim 1, wherein the heavy chain variable domain comprises the amino acid sequence of SEQ ID NO: 14.

4. The monoclonal antibody or antigen-binding fragment thereof according to claim 1, wherein:
(a) the light chain variable domain comprises the amino acid sequence of SEQ ID NO: 13 and
(b) the heavy chain variable domain comprises the amino acid sequence of SEQ ID NO: 14.

5. The monoclonal antibody according to any one of claims 1-4, wherein the antibody that specifically binds to AXL is a full-length IgG antibody.

6. The monoclonal antibody according to claim 5, wherein the full-length IgG antibody is of human IgG1, IgG2, IgG3 or IgG4 isotype.

7. The monoclonal antibody according to claim 6, wherein the antibody comprises the mutations S239D and I332E according to the EU numbering scheme for amino acids of antibodies.

8. The monoclonal antibody according to claim 6, wherein the antibody comprises the deletion 446G and 447K according to the EU numbering scheme for amino acids of antibodies in the CH3 region.

9. The monoclonal antibody according to claim 1, comprising a light chain comprising the amino acid sequence of SEQ ID NO: 15.

10. The monoclonal antibody according to claim 1, comprising a heavy chain comprising an amino acid sequence that is selected from the group: SEQ ID NO: 16 or SEQ ID NO: 17.

11. The monoclonal antibody according to claim 1, comprising:
(i) (a) a light chain comprising the amino acid sequence of SEQ ID NO: 15, and
(b) a heavy chain comprising the amino acid sequence of SEQ ID NO: 16; or
(ii) (a) a light chain comprising the amino acid sequence of SEQ ID NO: 15, and
(b) a heavy chain comprising the amino acid sequence of SEQ ID NO: 17.

12. The nucleic acid that encodes the antibody or antigen-binding fragment thereof according to any one of claims 1-11.

13. The nucleic acid according to claim 12, wherein the nucleic acid is DNA.

14. An expression vector comprising the nucleic acid according to any one of claims 12-13.

15. A method for producing a host cell to produce the antibody or antigen-binding fragment thereof according to any one of claims 1-11, comprising cell transformation by the vector according to claim 14.

16. A host cell for producing the antibody or antigen-binding fragment thereof according to any one of claims 1-11, comprising the nucleic acid according to any one of claims 12-13.

17. A method for producing the antibody or antigen-binding fragment thereof according to any one of claims 1-11, comprising culturing the host cell according to claim 16 in a culture medium under conditions sufficient to produce said antibody or antigen-binding fragment thereof, followed by isolation and purification of the resulting antibody or antigen-binding fragment thereof.

18. A pharmaceutical composition for treating an AXL-mediated disease or disorder comprising the antibody or antigen-binding fragment thereof according to any one of claims 1-11 in a therapeutically effective amount in combination with one or more pharmaceutically acceptable excipients.

19. The pharmaceutical composition according to claim 18, wherein the AXL-mediated disease or disorder is selected from the group: non-small cell lung cancer, non-small cell lung cancer with a EGFR mutation, breast carcinoma, HER2-positive breast cancer, four time-negative breast cancer, triple-negative breast cancer (TNBC), ovarian cancer, platinum-resistant ovarian cancer, prostate cancer, docetaxel-resistant prostate cancer, endometrial cancer and uterine sarcoma, endometrioid adenocarcinoma, uterine serous carcinoma, skin melanoma, neuroblastoma, glioblastoma, head and neck squamous cell carcinoma, stomach cancer, renal cell carcinoma, urothelial carcinoma, colorectal cancer, colon cancer, hepatocellular carcinoma, pancreatic cancer, biliary cancer, malignant neoplasm of the extrahepatic bile duct, intrahepatic bile duct cancer, malignant neoplasms of the gallbladder, osteosarcoma, Ewing sarcoma, neoplasms with high-level AXL expression, chronic myeloid leukemia, chronic lymphocytic leukemia, acute myeloid leukemia, acute lymphoblastic leukemia, chronic liver disease, non-alcoholic steatohepatitis, Alzheimer's disease or idiopathic pulmonary fibrosis.

20. A pharmaceutical composition for treating an AXL-mediated disease or disorder, comprising the antibody or antigen-binding fragment thereof according to any one of claims 1-11 and at least one other therapeutically active compound.

21. The pharmaceutical composition according to claim 20, wherein the AXL-mediated disease or disorder is selected from the group: non-small cell lung cancer, non-small cell lung cancer with a EGFR mutation, breast carcinoma, HER2-positive breast cancer, four time-negative breast cancer, triple-negative breast cancer (TNBC), ovarian cancer, platinum-resistant ovarian cancer, prostate cancer, docetaxel-resistant prostate cancer, endometrial cancer and uterine sarcoma, endometrioid adenocarcinoma, uterine serous carcinoma, skin melanoma, neuroblastoma, glioblastoma, head and neck squamous cell carcinoma, stomach cancer, renal cell carcinoma, urothelial carcinoma, colorectal cancer, colon cancer, hepatocellular carcinoma, pancreatic cancer, biliary cancer, malignant neoplasm of the extrahepatic bile duct, intrahepatic bile duct cancer, malignant neoplasms of the gallbladder, osteosarcoma, Ewing sarcoma, neoplasms with high-level AXL expression, chronic myeloid leukemia, chronic lymphocytic leukemia, acute myeloid leukemia, acute lymphoblastic leukemia, chronic liver disease, non-alcoholic steatohepatitis, Alzheimer's disease or idiopathic pulmonary fibrosis.

22. The pharmaceutical composition according to any one of claims 20-21, wherein the other therapeutically active compound is an antibody, a small molecule, a hormone therapy agent or combination thereof.

23. A method of treatment of an AXL-mediated disease or disorder, comprising administering to a subject in need of such treatment the antibody or antigen-binding fragment thereof according to any one of claims 1-11 or the pharmaceutical composition according to any one of claims 18-22 in a therapeutically effective amount.

24. A method of treatment of an AXL-mediated disease or disorder according to claim 23, comprising administering to a subject in need of such treatment the antibody or antigen-binding fragment thereof according to any one of claims 1-11 and at least one other therapeutically active compound in a therapeutically effective amount.

25. The method of treatment of the disease or disorder according to any one of claims 23-24, wherein the AXL-mediated disease or disorder is selected from the group: non-small cell lung cancer, non-small cell lung cancer with a EGFR mutation, breast carcinoma, HER2-positive breast cancer, four time-negative breast cancer, triple-negative breast cancer (TNBC), ovarian cancer, platinum-resistant ovarian cancer, prostate cancer, docetaxel-resistant prostate cancer, endometrial cancer and uterine sarcoma, endometrioid adenocarcinoma, uterine serous carcinoma, skin melanoma, neuroblastoma, glioblastoma, head and neck squamous cell carcinoma, stomach cancer, renal cell carcinoma, urothelial carcinoma, colorectal cancer, colon cancer, hepatocellular carcinoma, pancreatic cancer, biliary cancer, malignant neoplasm of the extrahepatic bile duct, intrahepatic bile duct cancer, malignant neoplasms of the gallbladder, osteosarcoma, Ewing sarcoma, neoplasms with high-level AXL expression, chronic myeloid leukemia, chronic lymphocytic leukemia, acute myeloid leukemia, acute lymphoblastic leukemia, chronic liver disease, non-alcoholic steatohepatitis, Alzheimer's disease or idiopathic pulmonary fibrosis.

26. The method of treatment of a disease or disorder according to any one of claims 24-25, wherein the other therapeutically active compound is an antibody, a small molecule, a hormone therapy agent or combination thereof.

27. Use of the antibody or antigen-binding fragment thereof according to any one of claims 1-11 or the pharmaceutical composition according to any one of claims 18-22 for treating an AXL-mediated disease or disorder in a subject in need of such treatment.

28. The use according to claim 27 of the antibody or antigen-binding fragment thereof according to any one of claims 1-11 and at least one other therapeutically active compound for treating an AXL-mediated disease or disorder in a subject in need of such treatment.

29. The use according to claim 28, wherein the AXL-mediated disease or disorder is selected from the group: non-small cell lung cancer, non-small cell lung cancer with a EGFR mutation, breast carcinoma, HER2-positive breast cancer, four time-negative breast cancer, triple-negative breast cancer (TNBC), ovarian cancer, platinum-resistant ovarian cancer, prostate cancer, docetaxel-resistant prostate cancer, endometrial cancer and uterine sarcoma, endometrioid adenocarcinoma, uterine serous carcinoma, skin melanoma, neuroblastoma, glioblastoma, head and neck squamous cell carcinoma, stomach cancer, renal cell carcinoma, urothelial carcinoma, colorectal cancer, colon cancer, hepatocellular carcinoma, pancreatic cancer, biliary cancer, malignant neoplasm of the extrahepatic bile duct, intrahepatic bile duct cancer, malignant neoplasms of the gallbladder, osteosarcoma, Ewing sarcoma, neoplasms with high-level AXL expression, chronic myeloid leukemia, chronic lymphocytic leukemia, acute myeloid leukemia, acute lymphoblastic leukemia, chronic liver disease, non-alcoholic steatohepatitis, Alzheimer's disease or idiopathic pulmonary fibrosis.

30. The use according to claim 28, wherein the other therapeutically active compound is an antibody, small molecule, hormone therapy agent, or any combination thereof.
